(19) United States
(12) Patent Application Publication
(10) Pub. No.: US 2004/0219573 A1
(43) Pub. Date: Nov. 4, 2004

(54) Screening method using BNPI and DNPI

(75) Inventors: Eberhard Weihe, Marburg (DE); Martin K.H. Schafer, Marburg (DE); Annette Bieller, Coelbe-Buergeln (DE)

Correspondence Address: CROWELL & MORING LLP, INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/734,731

(22) Filed: Dec. 15, 2003

Related U.S. Application Data
(63) Continuation of application No. PCT/EP02/06484, filed on Jun. 13, 2002.

(30) Foreign Application Priority Data
Jun. 13, 2001 (DE) 101 28 541.8

(51) Int. Cl. C12Q 1/68; G01N 33/53; G01N 33/567

(52) U.S. Cl. 435/6; 435/7.2

(57) ABSTRACT

Methods for detecting pain-regulating substances using BNPI and/or DNPI and the use of compounds thereby identified, active compounds which bind to BNPI and/or DNPI, antibodies directed against BNPI and/or DNPI, antisense nucleotides against BNPI and/or DNPI, or BNPI and/or DNPI or part proteins thereof, and corresponding polynucleotides for pharmaceutical formulations for pain therapy and diagnostic agents are provided. Certain nucleotide sequences, proteins and part proteins are also provided.

### CROSS-REFERENCE TO RELATED APPLICATIONS

This application is a continuation of International Patent Application No. PCT/EP02/06484, filed Jun. 18, 2002, designating the United States of America, and published in German as WO 02/101394, the entire disclosure of which is incorporated herein by reference. Priority is claimed based on Federal Republic of Germany Patent Application No. DE 101 28 541.8, filed Jun. 13, 2001.

### FIELD OF THE INVENTION

The invention relates to methods for detecting pain-regulating substances using BNPI and/or DNPI and the use of compounds thereby identified, active compounds which bind to BNPI and/or DNPI, antibodies directed against BNPI and/or DNPI, antisense nucleotides against BNPI and/or DNPI, or BNPI and/or DNPI or part proteins thereof, and corresponding polynucleotides for pharmaceutical formulations for pain therapy and diagnostic agents.

### BACKGROUND OF THE INVENTION

Various pharmaceutical formulations are available for pain therapy, e.g. acetylsalicylic acid, paracetamol, dipyrone, tramadol, morphine and fentanyl; however, substances such as amitryptiline and ketamine are also employed for treatment of pain patients. In spite of increasingly refined therapy plans, often no permanent improvement can be achieved for the patients. This is especially true in the case of chronic states of pain. With chronic pain, permanent changes to the involved nerve cells occurs, contributing to the problem.

Pain research in recent years has produced the fundamental finding that the development of chronic states of pain is based on plastic changes to the nervous system, in particular in the nociceptive neurons of the posterior root ganglia and the neurons in the region of the dorsal horns of the spinal cord (as an overview see: Coderre et al. 1993; Zimmermann & Herdegen, 1996). The neuronal plasticity is accompanied by changes in the expression of certain genes and leads to a long-lasting change in the phenotype of the neurons affected. The concept of neuronal plasticity has hitherto been applied to development, learning and regeneration processes. Recent findings from pain research show that this concept also intervenes in pathophysiological processes (Tölle, 1997).

The chronic development of pain has already been characterized relatively well at a phenomenological level in animal studies. Induction of chronic states of pain leads to the following changes:

Increased sensitivity and reduced stimulus threshold of peripheral nociceptors

Activation of so-called silent nociceptors

Reorganization of receptive fields

Increase in excitability in the spinal cord.

These plastic changes have been described both for the primary afferent fibers which occur in the ganglia and for the subsequent neurons located in the spinal cord, and are also assumed to occur supraspinally, e.g., in the thalamus. Like the mechanisms described for learning and memory processes, it is to be assumed that a specific gene program which comprises coordinated regulation of relevant genes proceeds in the cells involved. Expression of these genes contributes decisively to the pathophysiological manifestation of chronic pain.

The starting point of the invention was therefore the identification of such pain-regulated genes which are modified in their expression under pain conditions and are therefore probably involved, via their regulation connections, in the development and processing of chronic pain.

Regulatory functions have been detected for a number of known genes in various pain models (see Table 1). Certain genes for neurotransmitters (substance P, CGRP), receptors (substance P receptor, μ-, κ-, δ-opiate receptors, NMDA receptor) and transcription factors (cJun, JunB, cFos or Krox24) are examples thereof. The fact that the receptors mentioned are already used as molecular targets for the development of new analgesics (Dickenson, 1995) provides a clear indication that the identification of new pain-regulated genes is of great interest for the development of analgesics. Screening methods are of particular importance for such identification. The central idea is to interrupt the development or persistence of pain, particularly chronic pain, by influencing the function of those proteins which are formed to an increased or decreased extent in states of pain.

TABLE 1
Regulation of known genes/gene products in pain animal models

| Gene (product) | Reg | Tissue/cell | Model | Literature |
|---|---|---|---|---|
| (a) Neurotransmitters | | | | |
| CGRP | [symbol] | SC dorsal | UV | Gillardon F et al. |
| | | horn | irradiation of | (1992) |
| | | | the skin | Ann NY Acad |
| | | | | Sci657: 493-96 |
| Preprotachykinin & | [symbol] | DRG | Monoarthritis | Donaldson LF et |
| CGRP-mRNA | | | | al. (1992) |
| | | | | Mol Brain Res |
| | | | | 16: 143-49 |
| Preprotachykinin- | [symbol] | SC dorsal | Formalin | Noguchi & Ruda |
| mRNA | | horn | | (1992) |
| | | | | J Neurosci |
| | | | | 12: 2563-72 |
| Prodynorphin | [symbol] | Spinal cord | Exp. arthritis | Hollt et al. (1987) |
| mRNA | | | | Neurosci Lett |
| | | | | 96: 247-52 |
| Dynorphin prot. | [symbol] | Spinal cord | Formalin | Ruda et al. (1988) |
| | | | | PNAS 85: 622-26 |
| Substance P | [symbol] | Nociceptors | Exp. arthritis | Levine JD et al. |
| | | | | (1984) |
| | | | | Science 226: 547-49 |
| (b) Neurotrophins | | | | |
| BDNF mRNA & | [symbol] | DRG: | intrathecal | Michael GC et al. |
| immune reactivity | | trkA+ cells | NGF inj. | (1997) |
| | | | | J Neurosci 17: |
| | | | | 8476-90 |
| (c) Receptors | | | | |
| μ-, κ-, δ-binding | [symbol] | SC dorsal | Monoarthritis | Besse D et al. |
| | | horn | | (1992) |
| | | | | Eur J Pharmacol |
| | | | | 223: 123-31 |
| μ-Opiate receptor | [symbol] | DRG | Carrageenan | Ji R-R et al. (1995) |
| immune reactivity | | | ind. | J Neurosci |
| | | | inflammation | 15: 8156-66 |
| κ- & δ-opiate rec.- | [symbol] | DRG | Carrageenan | Ji R-R et al. (1995) |
| mRNA | | | ind. | J Neurosci |
| | | | inflammation | 15: 8156-66 |
| κ- & μ-opiate | [symbol] | SC dorsal | Monoarthritis | Maekawa K et al. |
| receptor-mRNA | | horn | | (1995) |
| | | | | Pain 64: 365-71 |
| CCK_{B}-rec. mRNA | [symbol] | DRG | Axotomy | Zhang X et al. (1993) |
| | | | | Neuroscience |
| | | | | 57: 227-233 |
| NMDA-R1-mRNA | [symbol] | SC dorsal | CFA-induced | Kus L et al. (1995) |
| | | horn | inflammation | Neuroscience |
| | | laminae I & II | | 68: 159-65 |
| (d) Enzymes | | | | |
| NADPH-diaphorase | [symbol] | SC dorsal | Ischiaticus | Fiallos-Estrada et |
| activity | | horn | transection | al. (′93) |
| | | | | Neurosci. Lett |
| | | | | 150: (169-73) |
| NADPH-diaphorase | [symbol] | Spinal cord | Formalin | Solodkin et al. |
| | | | | 1992 |
| | | | | Neurosci 51: 495-99 |
| NO synthetase | [symbol] | DRG | Axotomy | Verge VMK et al. |
| mRNA | | | | (1992) |
| | | | | PNAS 89: 11617-62 |
| NO synthetase | [symbol] | SC dorsal | Formalin | Herdegen et al. |
| protein | | horn | | (1994) |
| | | | | Mol Brain Res |
| | | | | 22: 245-58 |
| NO synthetase | [symbol] | DRG | Ischiaticus | Fiallos-Estrada et |
| immune reactivity | | | transection | al. (′93) |
| | | | | Neurosci Lett |
| | | | | 150: 169-73 |
| (e) Signal cascades | | | | |
| rap1A, rap1B, H-ras | [symbol] | Spinal cord | Formalin | Urayama O et al. |
| mRNA | | | | (1997) |
| | | | | Mol Brain Res |
| | | | | 45: 331-34 |
| PKC-binding | [symbol] | SC dorsal | CFA-induced | Tolle TR et al. (82) |
| | | horn | monoarthritis | J Neurol |
| | | | | 242(S2): 135 |
| (f) Transcription f. | | | | |
| cFOS | [symbol] | Spinal cord | Noxic | Hunt SP et al. |
| | | | stimulation | (1987) |
| | | | | Nature 328: 632-34 |
| cJun, JunB, cFOS | [symbol] | SC dorsal | Formalin | Herdegen T et al. |
| Krox24 | | horn | | (1994) |
| | | | | Mol Brain Res |
| | | | | 22: 245-48 |

SC, spinal cord;
DRG, dorsal root ganglia;
CFA, complete Freund's adjuvant;
NGF, nerve growth factor

### SUMMARY OF THE INVENTION

A primary object of the invention is to provide a screening method for identification of substances relevant in pain, in particular pain-regulating substances. The invention therefore relates to a method for detecting pain-regulating substances with the following method steps:

(a) incubating under suitable conditions of a test substance with a biomolecule from group I: the protein BNPI or DNPI and/or a protein according to one of FIGS. 1b), 1d), 1f), 1h), 2b), 2d) or 2f) (SEQ ID NOS 2, 4, 6, 8, 10, 12 or 14) and/or a protein which is at least 90% homologous thereto and/or a protein encoded by a polynucleotide according to one of FIGS. 1a), 1c), 1e), 1g), 2a), 2c) or 2e) (SEQ ID NOS 1, 3, 5, 7, 9, 11 or 13) or a polynucleotide which is at least 90% homologous thereto, and/or a protein encoded by a nucleic acid which binds under stringent conditions to a polynucleotide according to one of FIGS. 1a), 1c), 1e), 1g), 2a), 2c) or 2e) (SEQ ID NOS 1, 3, 5, 7, 9, 11 or 13) or antisense polynucleotides thereof, or a part protein of one of the abovementioned proteins which is at least 10, preferably at least 15, in particular at least 20 amino acids long and/or a cell and/or a preparation from a cell which has synthesized at least one of the abovementioned proteins and part proteins,

(b) measuring the binding of the test substance to the protein or part protein synthesized by the cell or measurement of at least one of the functional parameters modified by the binding of the test substance to the protein or part protein.

This novel screening method is based on the detection of a potential pain activity of a substance by its interaction with a pain-regulated protein structure, in particular, BNPI or DNPI or related structures.

The term pain-regulating here relates to a potential regulating influence on the physiological pain event, in particular to an analgesic action. The term substance includes any compound suitable as an active compound for a pharmaceutical formulation, in particular, low molecular weight active compounds, but also others, such as nucleic acids, fats, sugars, peptides or proteins, such as antibodies.

Incubation under suitable conditions is to be understood here as meaning that the substance to be investigated can react with the cell or the corresponding preparation in an aqueous medium a defined time before the measurement. The aqueous medium can be temperature-controlled here, for example between 4° C. and 40° C., and is preferably at room temperature or at 37° C. The incubation time can be varied between a few seconds and several hours, depending on the interaction of the substance with the part protein or protein. Incubation times of between 1 min and 60 min are preferred. The aqueous medium may comprise suitable salts and/or buffer systems, such that, for example, a pH of between 6 and 8, preferably pH 7.0-7.5, is maintained in the medium during the incubation. Suitable substances, such as coenzymes, nutrients etc., may also be added to the medium. The suitable conditions can easily be optimized by persons skilled in the art as a function of the interaction of the substance to be investigated of the substance with the part protein or protein on the basis of experience, the literature or a few simple preliminary experiments, in order to obtain the clearest possible measurement value in the method.

A cell which has synthesized a particular part protein or protein is a cell which has already expressed this part protein or protein endogenously or one which has been modified by genetic engineering such that it expresses this part protein or protein and accordingly contains the part protein or protein before the start of the method according to the invention. The cells can be from immortalized cell lines or can be native cells originating and isolated from tissues, the cell union usually being broken down. The preparation from these cells comprises, in particular, homogenates from the cells, the cytosol, a membrane fraction of the cells with membrane fragments, a suspension of isolated cell organelles, etc.

The proteins and part proteins listed here have been identified in the context of this invention as regulated by pain or distributed in a pain-relevant manner by inducing pain in an animal and, after an appropriate period of time, comparing the expression pattern of the animal with that of a control animal without pain-inducing measures by sections in the spinal cord. Those found here as modified in expression are BNPI and, in particular in respect of pain-relevant distribution, DNPI.

The species from which these proteins originate is irrelevant for the functioning of the method, but it is preferable to use the human, mouse or rat variants. BNPI and DNPI are known in respect of the coding DNA sequence and the amino acid sequence and are also described in their general function. BNPI, the “brain Na+ dependent inorganic phosphate cotransporter”, is described in WO 96/34288 and DNPI, the “differentiation-associated Na+ dependent inorganic phosphate cotransporter”, has been described by Aihara et al. (2000) in J. Neurochem. 74, 2622-2625.

However, neither of these transporters has hitherto been connected with pain and, in particular, pain regulation in the prior art. Since the identification of the proteins took place here via a modification of the expression or via the expression distribution in an in vivo pain model, for future pharmaceutical formulations using these proteins the screening method according to the invention derived therefrom has the considerable advantage not only of being built up on theoretical considerations but presumably of having a strong in vivo relevance. Since with this method the interaction of substances with proteins and peptides not hitherto used in the pain sector is rendered possible as a standard for detecting pain-regulating substances, pain-relevant substances which would not have emerged in the methods known hitherto in the prior art using other peptides or proteins are now possibly detected with this method. This is also a considerable advantage of the new method according to the invention.

The standard by which the method allows the detection of interesting substances is either the binding to the protein or part protein, which can be detected e.g. by displacement of a known ligand or the extent of the substance bound, or the modification of a functional parameter due to the interaction of the substance with the part protein or protein. This interaction can lie, in particular, in a regulation, inhibition and/or activation of receptors, ion channels and/or enzymes, and modified functional parameters can be, for example, the gene expression, the ionic medium, the pH or the membrane potential, or the modification of the enzyme activity or the concentration of the second messenger.

To explain the invention, in addition to the explanations given for terms in the general text, further definitions are given below in order to clarify how certain terms used in the claims in particular are to be understood and interpreted in the context of this invention.

Substance: This means a chemical compound. In one sense, these are compounds which can potentially display an action in the body, low molecular weight active compounds, nucleic acids, fats, sugars, peptides or proteins, low molecular weight active compounds in particular here.

Pain-regulating: In the context of the invention, pain-regulating means that the substance directly or indirectly influences the perception of pain. Substances with a natural analgesic action are of particular relevance.

Pain: In the context of the invention, pain means in particular a pain sensation, more precisely acute, chronic, neuropathic and inflammatory pain, including migraine, and in particular the pain includes the following types:

chronic pain, in particular musculoskeletal pain; neuropathic pain, in particular allodynic pain, mechanical hyperalgesia or diabetic neuropathy; visceral pain, cerebral pain, peripheral pain or inflammation-related pain, in particular peripheral inflammation pain; and migraine, cluster headache or pain with trigeminus neuralgia.

Incubation: Incubation means the procedure in which a biological object for investigation, for example a cell or a protein, is introduced into and left in a temperature-controlled medium, such as in an incubating cabinet or on a water-bath. Suitable conditions for incubation under physiological conditions (e.g., 37° C., pH 7.2) or conditions under which an optimum measurement in the method is possible.

Cell: The cell is a self-regulating, open system which is in a flow equilibrium with its environment by permanent exchange of matter and has its own metabolism and ability to multiply. The cell can be cultured separately or can be part of a tissue, in particular from an organ, and can exist there individually or also in a cell union.

Preparation from a cell: This means preparations which are prepared by means of chemical, biological, mechanical or physical methods causing a change in the cell structure. For example, membrane fragments, isolated cell compartments, isolated cytosol, or homogenate obtained from tissue may be included.

Peptide: Means a compound of amino acids linked to chains via peptide bonds. An oligopeptide consists of between 2 and 9 amino acids and a polypeptide of between 10 and 100 amino acids.

Protein: Means a compound of more than 100 amino acids linked as a chain via peptide bonds, under certain circumstances with a defined spatial structure.

Part protein: Means a compound of more than 10 amino acids linked as a chain via peptide bonds, under certain circumstances with a defined spatial structure, but cut out or selected from a defined protein. A part protein can be a peptide.

PIM1-kinase: PIM3-kinase: A proto-oncogene and serine-threonine kinase.

Polynucleotide: The underlying nucleotide is in principle a base unit of nucleic acids which consists of nuclein base, pentose and phosphoric acid. This corresponds to a high molecular weight polynucleotide of several nucleotides linked to one another via phosphoric acid-pentose esterification. However, the invention also contemplates modified polynucleotides, which retain the base sequence but have a modified backbone instead of phosphoric acid-pentose.

Similar to the extent of at least 90 (95, 97)%: This means that in their coding region the polynucleotides referred to are at least 90% (95%, 97%) identical to the reference (figure, SEQ. ID NO., etc.) with respect to the base sequence, and in their primary structure, the sequence of amino acids, the peptides and proteins referred to are identical to the extent of at least 90% (95%, 97%) to the reference.

Gene: The term gene describes a genome section with a defined nucleotide sequence which contains the information for synthesis of an m- or pre-mRNA or another RNA (e.g. tRNA, rRNA, snRNA etc). It consists of coding and non-coding sections.

Gene fragment: Means a nucleic acid section which comprises a part region of a gene in its base sequence.

Binding to the peptide, part protein or protein: This means interaction between a substance and peptide, part protein or protein which leads to fixing.

Functional parameters: This means measurement parameters of an experiment which correlate with the function of a protein (ion channel, receptor, enzyme).

Manipulated by genetic engineering: This means the manipulation of cells, tissues or organisms such that genetic material is introduced.

Expressed endogenously: This means the expression of a protein in a cell line under suitable culture conditions without this corresponding protein having been prompted to expression by manipulation by genetic engineering.

G protein: The means the internationally conventional abbreviation for a guanosine triphosphate (GTP)-binding protein which is activated as a signal protein by receptors coupled to G protein.

Reporter gene: This is a general term for genes of which the gene products can be detected easily with the aid of simple biochemical methods or histochemical methods, such as luciferase, alkaline phosphatase or green fluorescent protein (GFP).

(Recombinant) DNA construct: This is a general term for any type of DNA molecules which are formed by in vitro linking of DNA molecules.

Cloning vector: This is a general term for nucleic acid molecules which serve as carriers of foreign genes or parts of these genes during cloning.

Expression vector: This is a term for specially constructed cloning vectors which, after introduction into a suitable host cell, allow transcription and translation of the foreign gene cloned into the vector.

LTR sequence: This is an abbreviation for long terminal repeat, which is a general term for longer sequence regions which are located at both ends of a linear genome. These sequence regions occur, e.g., in the genomes of retroviruses and at the ends of eukaryotic transposons.

Poly A tail: This means the adenyl radicals or residues attached at the 3′ end of messenger RNA by polyadenylation (approx. 20-250).

Promoter sequence: A term for a DNA sequence region from where the transcription of a gene, i.e. the synthesis of the mRNA, is controlled.

ORI sequence: An abbreviation for origin of replication. The ORI sequence allows a DNA molecule to multiply as an autonomous unit in the cell.

Enhancer sequence: A term for relatively short, genetic elements, which in some cases occur as repetitions and which as a rule enhance the expression of some genes to a varying degree.

Transcription factor: A term for a protein which influences the transcription of a gene via binding to specific DNA sequences.

Culturing: This means keeping cells or tissue under suitable culture conditions.

Conditions which allow expression: This means the choice and use of culture conditions which allow expression of a protein of interest, which includes changes in temperature, changes of medium, addition of inducing substances and omission of inhibiting substances.

Incubation time: This means the duration of time for which cells or tissue are incubated, i.e. exposed to a defined temperature.

Selection pressure: The means the application of culture conditions which provide cells which have a particular gene product, the so-called selection marker, with a growth advantage.

Amphibia cell: A cell from an animal of the Amphibia class.

Bacteria cell: A cell which is to be assigned to the superkingdom of Eubacteria or Archaebacteria or originates from it.

Yeast cell: A cell which is to be assigned to the order of the Endomycetales or originates from it.

Insect cell: A cell which is to be assigned to the order of the Hexapoda or originates from it.

Native mammalian cell: A cell originating from a mammal which corresponds in its relevant features to the cell present in the organism.

Immortalized mammalian cell: A cell which has acquired, by the culture conditions applied or manipulation by genetic engineering, the property of dividing in the culture beyond the usual conventional frequency of division (approx. 100).

Labeled: This means rendered accessible to a detection reaction by appropriate modification or derivatization. For example radioactively, fluorescently or luminescently.

Ligand: A substance which binds to a molecule present in the body or a cell, specifically a receptor.

Displacement: The complete or partial removal of a ligand from its binding site.

Bound activity: A measurement value which correlates with the amount of ligand bound to a receptor. The value may be determined biochemically or physically.

Regulation: The inhibition or activation of a process which takes place as part of a regulating process.

Inhibition: Inhibition/reduction of a process as a special case of regulation.

Activation: Intensification of a process as a special case of regulation.

Receptors: In the broadest sense, this means all the molecules present in the pro- or eukaryotic organism which can bind to an active compound. In the narrower sense, this means membrane-bound proteins or complexes of several proteins which direct a change in the cell after binding an active compound.

Ion channels: Membrane-bound proteins or complexes of several proteins which permit cations or anions to pass through the membrane.

Enzymes: A term for proteins or complexes of an activating non-protein component with a protein having catalytic properties.

Gene expression (express/expressible): The translation of the genetic information of a gene into RNA (RNA expression) or into protein (protein expression).

Ionic medium: An ion concentration of one or more ions in a particular compartment.

Membrane potential: A potential difference over a membrane on the basis of an excess of cations on one side and anions on the other side of the membrane.

Change in enzyme activity: The inhibition or induction of the catalytic activity of an enzyme.

2nd messenger: A small molecule which, as a response to an extracellular signal, either is formed in the cytosol or migrates into the cytosol and thereby helps to transmit information to the inside of the cell, for example, cAMP, IP₃.

(Gene) probe: A term for any type of nucleic acids which enable detection of a gene or a particular DNA sequence. By derivatization of the gene probe (e.g. using biotin, magnetic beads or digoxinin), DNA molecules can furthermore be drawn out of a mixture. Cloned genes, gene fragments, chemically synthesized oligonucleotides and also RNA, which is usually radioactively labeled, are used as probes.

DNA: An international term for deoxyribonucleic acid.

Genomic DNA: A general term for the DNA originating from the cell nucleus of a cell in eukaryotic organisms.

cDNA: An abbreviation for complementary DNA. This term for the single-or double-stranded DNA copy of an RNA molecule.

cDNA bank/library: A term for a collection of arbitrarily cloned cDNA fragments which, taken together, represent the entirety of all the RNA synthesized by a cell or a tissue.

cDNA clone: A term for a population of genetically uniform cells which are derived from a single cell such that this cell contains an artificially introduced cDNA fragment.

Hybridization: The formation, effected by base pairing, of a double-stranded nucleic acid molecule from two separate single strands.

Stringent conditions: Conditions under which only perfectly base-paired nucleic acid strands are formed and remain stable.

Isolate: To discover and separate a molecule from a mixture.

DNA sequencing: The determination of the sequence of from bases in a DNA molecule.

Nucleic acid sequence: A term for the primary structure of a DNA molecule, i.e. the sequence of the individual bases from which a DNA molecule is composed.

Gene-specific oligonucleotide primer: Oligonucleic acids, preferably nucleic acid fragments 10-40 bases long, which, in their base composition, allow a stringent hybridization to the gene sought or the cDNA sought.

Determination of oligonucleotide primers: A manual or computer-assisted search of oligonucleotides for a given DNA sequence which are of optimum suitability for a hybridization and/or a polymerase chain reaction.

PCR: An abbreviation for polymerase chain reaction. The PCR is an in vitro process for selective concentration of nucleic acid regions of defined length and defined sequence, especially from a mixture of nucleic acid molecules.

DNA template: A nucleic acid molecule or a mixture of nucleic acid molecules from which a DNA section is multiplied with the aid of the PCR (see above).

RNA: An internationally common abbreviation for ribonucleic acids.

mRNA: An internationally common abbreviation for messenger ribonucleic acids which are involved in transfer of the genetic information from the nucleus into the cell and contain information for the synthesis of a polypeptide or a protein.

Antisense polynucleotide: A molecule comprising several natural or modified nucleic acids, the base sequence of which is complementary to the base sequence of a part region of an RNA which occurs in nature.

PNA: An internationally common abbreviation for peptidic nucleic acids. Peptidically linked amino acids form a chain, and different bases are linked so that the molecule is capable of hybridization with DNA or RNA.

Sequence: A sequence of nucleotides or amino acids. In the specific context of this invention, this means the nucleic acid sequence.

Ribozyme: A term for a catalytically active ribonucleic acid (e.g. ligase, endonuclease, polymerase, exonuclease).

DNA enzyme: A term for a DNA molecule which contains catalytic activity (e.g. ligase, endonuclease, polymerase, exonuclease).

Catalytic RNA/DNA: A general term for ribozymes or DNA enzymes (see above).

Adenovirus: A cytopathogenic virus which occurs in vertebrates.

Adeno-associated virus (AAV): Means a virus in the family of Parvoviruses. For effective multiplication of AAV, co-infection of the host cells with helper viruses (e.g. herpes, vaccinia or adeno-viruses) is necessary. The property of AAV of integrating into the host genome in a stable manner makes it of particular interest as a transduction vector for mammalian cells.

Herpes virus: A viral pathogen of herpes infection

Post-translational modification: A modification to proteins or polypeptides carried out after translation, which includes e.g. phosphorylation, glycosylation, amidation, acetylation or proteolysis.

Glycosylate: To append individual sugar molecules or whole sugar chains on to proteins.

Phosphorylate: Term for the appending of one or more phosphate radicals to a protein, preferably on to the OH groups of the amino acids serine, threonine or tyrosine.

Amidate: To convert a carboxyl function into an amide function, e.g. on the carboxy-terminal amino acid radical of a peptide or protein.

Provided with a membrane anchor: This means a post-translational modification of a protein or of another organic molecule such that, by appending a hydrophobic molecule, such as a fatty acid or a derivative thereof, it is anchored to the lipid double-layer membrane of cells.

Cleave: To cleave a peptide or protein into several sub-sequences.

Shorten: Shortening of a molecule consisting of several individual parts by one or more parts.

Antibodies: This means proteins, called immunoglobulins, which are soluble or bound to cell membranes and have a specific binding site for antigens.

Monoclonal antibodies: Antibodies which have an extremely high selectivity and are directed against a single antigenic determinant of an antigen.

Polyclonal antibodies: A mixture of antibodies directed against several determinants of an antigen.

Transgenic: This means genetically modified.

Non-human mammal: The entirety of mammals (class of Mammalia) with the exception of the human species.

Germ cell: A cell with a haploid genome which, by fusion with a second germ cell, renders possible the formation of a new organism.

Somatic cell: A diploid cell that is normally a constituent of an organism.

Chromosomal introduction: An intervention in the nucleotide sequence at the chromosomal level.

Genome: A general description of the entirety of all the genes in an organism.

Ancestor of the animal: An animal (the ancestor) which is related in a direct line with another animal (the descendant) in a natural or artificial manner by passing on its genetic material.

Expressible: A nucleic acid molecule is expressible if it contains the information for synthesis of a protein or polypeptide and is provided with appropriate regulatory sequences which allow synthesis of this protein or polypeptide in vitro or in vivo. If these prerequisites no longer exist, for example by intervention into the coding sequence, the nucleic acid molecule is no longer expressible.

Rodent: An animal from the order of the Rodentia, e.g. rat or mouse.

Substance identifiable as pain-regulating: A substance which, when introduced into a living organism, causes a change in behavior which one skilled in the art would deem pain-inhibiting (antinociceptive, antihyperalgesic or antiallodynic). In the case of the screening method, this relates to the fact that, during screening, the substance significantly, for example by 100%, exceeds the binding or interaction of the average substances tested due to stronger binding or inducement of a modification in a functional parameter.

Compound: Another name for a molecule consisting of several atoms, preferably a molecule identified by the method according to the invention.

Active compound: A compound which, when used on an organism, causes a change in this organism. In particular, this means molecules synthesized by organic chemistry which have a healing action on the organism. Molecules which bind to the proteins and peptides according to the invention are particularly preferred.

Low molecular weight: A molecule with a molecule weight of <2 kDa.

Pharmaceutical formulation: A substance corresponding to the definition in article 1 §2 of the Act on Circulation of Medical Preparations.

Diagnostic agent: A compound or method which can be used to diagnose a disease.

Treatment of pain: A method with the aim of alleviating or eliminating pain or inhibiting the expected occurrence of pain (pre-emptive analgesia).

Chronic pain: A pain sensation of long-lasting duration, often characterized in that it increases the pain sensitivity of the body beyond the point in time and location of the initial stimulus.

Gene therapy: Gene therapy refers to all methods which have the aim of causal treatment of genetic diseases by suitable modifications to the genome.

In vivo gene therapy: The introduction of genetic material into the living organism with the aim of gene therapy. A distinction can be made between somatic and germ path intervention, which takes place in one instance on diploid cells and in the other instance on haploid cells.

In vitro gene therapy: The introduction of genetic material into cells outside the human body with the aim of subsequently using these again for gene therapy by introduction into the human body.

Diagnostics: Methods for identifying a disease.

Investigation of activity: An investigation with the aim of investigating the activity of a compound after acting on a living organism.

In a preferred embodiment of the method, the cell is manipulated by genetic engineering before step (a) above. In this procedure, genetic material is introduced into the cell, in particular one or more polynucleotide sequences. In a variant of this embodiment which is even more preferred, the manipulation by genetic engineering permits the measurement of at least one of the functional parameters modified by the test substance. In this embodiment, prerequisites under which the modification of a functional parameter can be measured at all or in an improved manner are created by manipulation by genetic engineering. It is particularly preferable here for a form of a G protein which is not expressed endogenously in the cell to be expressed or a reporter gene to be introduced by the manipulation by genetic engineering. This is to be understood, in particular, as meaning the introduction into the cell, by genetic engineering, of a G protein (GTP-binding protein) which is not present endogenously or is not expressed physiologically, for example the introduction of a chimeric G protein which allows a modification of the signal path or of a promiscuous G protein which binds very readily. The introduction of a reporter gene in turn allows the measurement of an (extracellularly triggered) induced expression of the gene product.

In another preferred embodiment, the cell is manipulated by genetic engineering such that the cell contains at least one polynucleotide according to one of FIGS. 1a), 1c), 1e), 1g), 2a), 2c) or 2e) (SEQ ID NOS 1, 3, 5, 7, 9, 11 or 13) or a polynucleotide which is similar thereto to the extent of at least 90%. The achievement of this can be, for example, that a part protein or protein which is not expressed endogenously in the cell or preparation used in the method is synthesized by the cell. It is particularly preferable here for the polynucleotide to be contained in a recombinant DNA construct. A (recombinant) DNA construct is understood as meaning a DNA molecule prepared in vitro.

If the cell is manipulated by genetic engineering before step a) in the method, it is preferable for the cell to be cultured, after the manipulation by genetic engineering and before step a), under conditions which allow an expression, optionally under selection pressure. Culturing is understood as meaning keeping cells or tissue under conditions which ensure survival of the cells or their subsequent generation. The conditions should be chosen here such that an expression of the material inserted by the manipulation by genetic engineering is rendered possible. For this, the pH, oxygen content and temperature should be kept at the physiological values and sufficient nutrients and necessary cofactors should be added. The selection pressure allows only the cells in which the manipulation by genetic engineering was at least partly successful to be cultured further. This includes, for example, introduction of an antibiotic resistance via the DNA construct.

It is particularly preferable in the method according to the invention for the cell used to be an amphibia cell, bacterial cell, yeast cell, insect cell or an immortalized or native mammalian cell. Examples for amphibia cells are Xenopus oocytes, for bacteria cells E. coli cells, for yeast cells those also Saccharomyces cerevisiae, for insect cells Sf9 cells, for immortalized mammalian cells HeLa cells and for native mammalian cells the CHO (Chinese hamster ovary) cell.

In a preferred measurement method for determination of the binding of the substance to part protein or protein in the method according to the invention, the measurement of the binding is carried out via the displacement of a known labeled ligand of the part protein or protein and/or via the activity bound thereto from a labeled test substance. A ligand here is a molecule which binds to the protein or part protein with a high specificity and is displaced from the binding site by a substance to be tested which also binds. Labeling is to be understood as meaning an artificial modification to the molecule which facilitates detection. Examples are radioactive, fluorescent or luminescent labeling.

In another preferred measurement method for determination of the modification of the functional parameter induced by the binding of the substance to the part protein or protein in the method according to the invention, measurement of at least one of the functional parameters modified by the test substance is carried out via measurement of the regulation, inhibition and/or activation of receptors, ion channels and/or enzymes, in particular via measurement of the modification in gene expression, the ionic medium, the pH or the membrane potential, via the modification in the enzyme activity or the concentration of the 2nd messenger. This includes on the one hand measurement of the action of the substance directly via influencing of receptors, ion channels and/or enzymes, and on the other hand, as examples which are preferably to be measured, measurement of parameters which are modified, such as gene expression, ionic medium, pH, membrane potential, enzyme activity or concentration of the 2nd messenger. Ionic medium is understood here as meaning, in particular, the concentration of one or more ions in a cell compartment, in particular the cytosol, membrane potential is understood here as meaning the charge difference between two sides of a biomembrane, and 2nd messenger is understood here as meaning messenger substances of the intracellular signal path, such as e.g. cyclic AMP (cAMP), inosotol triphosphate (IP3) or diacylglycerol (DAG).

This or these method(s) include the use of part proteins and in particular proteins with a known sequence and function, without a function in pain being known for these in the prior art.

A further particularly preferred embodiment is a method according to the invention in which a first of the methods according to the invention described hitherto is coupled with a second of the methods according to the invention described hitherto such that the measurement values and results of the first method in respect of the substance to be measured are compared with the measurement values and results of the second method in respect of the substance to be measured, characterized in that in one of the two methods, called the main method in the following, in step (a) the substance to be tested is incubated

either

with a biomolecule from group II: the protein BNPI and/or a protein according to one of FIGS. 1b), 1d), 1f) or 1h) (SEQ ID NOS 2, 4, 6 or 8) and/or a protein which is similar to one of these abovementioned proteins to the extent of at least 90% and/or a protein for which a polynucleotide according to one of FIGS. 1a), 1c), 1e) or 1g) (SEQ ID NOS 1, 3, 5 or 7) or a polynucleotide which is similar thereto to the extent of at least 90% codes, and/or a protein which is coded by a nucleic acid which binds under stringent conditions to a polynucleotide according to one of FIGS. 1a), 1c), 1e) or 1g) (SEQ ID NOS 1, 3, 5 or 7) or antisense polynucleotides thereof, or a part protein of one of the abovementioned proteins which is at least 10, preferably at least 15, in particular at least 20 amino acids long and/or a cell and/or a preparation from such a cell which has synthesized at least one of the abovementioned proteins and/or part proteins,

or

with a biomolecule from group III: the protein DNPI and/or a protein according to one of FIGS. 2b), 2d) or 2f) and/or a protein which is similar to one of these abovementioned proteins to the extent of at least 90% and/or a protein for which a polynucleotide according to one of FIGS. 2a), 2c) or 2e) or a polynucleotide which is similar thereto to the extent of at least 90% codes, and/or a protein which is coded by a nucleic acid which binds under stringent conditions to a polynucleotide according to one of FIGS. 2a), 2c) or 2e) or antisense polynucleotides thereof, or a part protein of one of the abovementioned proteins which is at least 10, preferably at least 15, in particular at least 20 amino acids long and/or a cell and/or a preparation from such a cell which has synthesized at least one of the abovementioned proteins and/or part proteins,

and

in that in the other of the two methods, called the secondary method in the following, in step (a) the substance to be tested is incubated with a biomolecule from group I or with a biomolecule from that group chosen from group II and group III from which the biomolecule with which the substance in the main method is incubated is not chosen.

This particularly preferred embodiment is to be understood as meaning in particular the combination of the measurement of binding to BNPI or biomolecules derived therefrom or the measurement of the modification of cellular parameters arising therefrom on the one hand and binding to DNPI and in each case biomolecules derived therefrom or the measurement of the modification of cellular parameters arising therefrom on the other hand, since precisely a comparison in view of the completely separate but closely adjacent distribution of the two channels in the tissue can give an important conclusion on physiological functions. Differential balancing of the data therefore allows, however, identification of substances of optimized pharmaceutical or medical activity.

A method according to the invention which is furthermore preferred is that wherein the pain regulated by the substance to be detected is chosen from:

chronic pain, in particular musculoskeletal pain; neuropathic pain, in particular allodynic pain, mechanical hyperalgesia or diabetic neuropathy; visceral pain, cerebral pain, peripheral pain or inflammation-related pain, in particular peripheral inflammation pain; and migraine, cluster headache or pain with trigeminus neuralgia.

The invention also provides a compound which is identifiable as a pain-regulating substance by a method according to the invention. Compound here relates in particular to low molecular weight active compounds, and also to peptides, proteins and nucleic acids. Identifiable here means that the compound has the feature that in the screening method according to the invention it binds significantly more strongly in respect of the binding, preferably twice as strongly, as the average of the substances to be tested or deviates significantly from the average of the substances to be tested in respect of the modification of the functional parameters. It is particularly preferable for the compound according to the invention to be a low molecular weight compound.

The invention also relates to the use of

a. a polynucleotide, preferably a DNA or RNA, which codes for BNPI or DNPI or a polynucleotide, preferably a DNA or RNA, which corresponds to the extent of at least 90%, preferably 95%, in particular to the extent of at least 97%, to one of the nucleotide sequences shown in FIGS. 1a), 1c), 1e), 1g), 2a), 2c) or 2e), (SEQ ID NOS 1, 3, 5, 7, 9, 11 or 13)

b. a polynucleotide, in particular an antisense polynucleotide or a PNA, preferably a DNA enzyme or ribozyme, a ribozyme or other DNA enzyme or a catalytic RNA or DNA, which has a nucleotide sequence which is capable of binding specifically to one of the polynucleotides listed under point a),

c. a vector containing a polynucleotide according to one of points a) or b), in particular an expression vector and/or in particular derived from a virus, for example the adenovirus, adeno-associated virus or herpes virus, and/or in particular containing at least one LTR, poly A, promoter and/or ORI sequence,

d. BNPI or DNPI and/or a protein according to one of FIGS. 1b), 1d), 1f), 1h), 2b), 2d) or 2f) (SEQ ID NOS 2, 4, 6, 8, 10, 12 or 14) and/or a protein which is similar to one of these abovementioned proteins to the extent of at least 90% and/or a protein for which a polynucleotide according to one of FIGS. 1a), 1c), 1e), 1g), 2a), 2c) or 2e) (SEQ ID NOS 1, 3, 5, 7, 9, 11 or 13) or a polynucleotide which is similar thereto to the extent of at least 90% codes, and/or a protein which is coded by a nucleic acid which binds under stringent conditions to a polynucleotide according to one of FIGS. 1a), 1c), 1e), 1g), 2a), 2c) or 2e) (SEQ ID NOS 1, 3, 5, 7, 9, 11 or 13) or antisense polynucleotides thereof or a part protein of one of the abovementioned proteins which is at least 10, preferably at least 15, in particular at least 20 amino acids long, wherein the protein or part protein has been optionally post-translationally modified, in particular glycosylated, phosphorylated, amidated, methylated, acetylated, ADP-ribosylated, hydroxylated, provided with a membrane anchor, cleaved or shortened,

e. an antibody, preferably a monoclonal or polyclonal antibody, against one of the proteins or part proteins according to point d),

f. a cell, preferably an amphibia cell, bacteria cell, yeast cell, insect cell or an immortalized or native mammalian cell, containing a polynucleotide according to one of points a) or b), a vector according to point c), a protein or part protein according to point d) or an antibody according to point e)

g. a compound according to one of claims 12 or 13 and/or

h. an active compound, preferably a low molecular weight active compound, which binds to a protein or part protein according to point a),

for the preparation of a pharmaceutical formulation for treatment of pain.

The use for treatment of chronic, in particular neuropathic or inflammation-related pain is particularly preferred.

The invention also provides the use of

a. a polynucleotide, preferably a DNA or RNA, which codes for BNPI or DNPI or a polynucleotide, preferably a DNA or RNA, which corresponds to the extent of at least 90%, preferably 95%, in particular to the extent of at least 97%, to one of the nucleotide sequences shown in FIGS. 1a), 1c), 1e), 1g), 2a), 2c) or 2e) (SEQ ID NOS 1, 3, 5, 7, 9, 11 or 13),

b. a polynucleotide, in particular an antisense polynucleotide or a PNA, preferably a DNA enzyme or ribozyme, a ribozyme or other DNA enzyme or a catalytic RNA or DNA, which has a nucleotide sequence which is capable of binding specifically to one of the polynucleotides listed under point a),

c. a vector containing a polynucleotide according to one of points a) or b), in particular an expression vector and/or in particular derived from a virus, for example the adenovirus, adeno-associated virus or herpes virus, and/or in particular containing at least one LTR, poly A, promoter and/or ORI sequence,

f. a cell, preferably an amphibia cell, bacteria cell, yeast cell, insect cell or an immortalized or native mammalian cell, containing a polynucleotide according to one of points a) or b) or a vector according to point c) for the preparation of a pharmaceutical formulation for use in gene therapy. It is particularly preferable here for the therapy to be in vivo or in vitro gene therapy. Gene therapy is understood as meaning a therapy form in which an effector gene, usually a protein, is expressed by introduction of nucleic acids into cells. A distinction is made in principle between in vivo and in vitro methods. In the case of in vitro methods, cells are removed from the organism and transfected ex vivo with vectors, in order to be subsequently introduced again into the same or into another organism. In the case of in vivo gene therapy, vectors, for example for combating tumours, are administered systemically (e.g. via the blood stream) or directly into the target tissue (e.g. into a tumour). It is furthermore preferable for the pharmaceutical formulation furthermore to be a pharmaceutical formulation for treatment of pain.

In the use in gene therapy, the use of a polynucleotide which is an antisense polynucleotide or PNA, or which is part of a ribozyme or other DNA enzyme or of a catalytic RNA or DNA is also preferred.

The invention also furthermore provides the use of

a. a polynucleotide, preferably a DNA or RNA, which codes for BNPI or DNPI or a polynucleotide, preferably a DNA or RNA, which corresponds to the extent of at least 90%, preferably 95%, in particular to the extent of at least 97%, to one of the nucleotide sequences shown in FIGS. 1a), 1c), 1e), 1g), 2a), 2c) or 2e) (SEQ ID NOS 1, 3, 5, 7, 9, 11 or 13),

b. a polynucleotide, in particular an antisense polynucleotide or a PNA, preferably a DNA enzyme or ribozyme, a ribozyme or other DNA enzyme or a catalytic RNA or DNA, which has a nucleotide sequence which is capable of binding specifically to one of the polynucleotides listed under point a),

c. a vector containing a polynucleotide according to one of points a) or b), in particular an expression vector and/or in particular derived from a virus, for example the adenovirus, adeno-associated virus or herpes virus, and/or in particular containing at least one LTR, poly A, promoter and/or ORI sequence,

d. BNPI or DNPI and/or a protein according to one of FIGS. 1b), 1d), 1f, 1h), 2b), 2d) or 2f) (SEQ ID NOS 2, 4, 6, 8, 10, 12 or 14) and/or a protein which is similar to one of these abovementioned proteins to the extent of at least 90% and/or a protein for which a polynucleotide according to one of FIGS. 1a), 1c), 1e), 1g), 2a), 2c) or 2e) (SEQ ID NOS 1, 3, 5, 7, 9, 11 or 13) or a polynucleotide which is similar thereto to the extent of at least 90% codes, and/or a protein which is coded by a nucleic acid which binds under stringent conditions to a polynucleotide according to one of FIGS. 1a), 1c), 1e), 1g), 2a), 2c) or 2e) (SEQ ID NOS 1, 3, 5, 7, 9, 11 or 13) or antisense polynucleotides thereof or a part protein of one of the abovementioned proteins which is at least 10, preferably at least 15, in particular at least 20 amino acids long, wherein the protein or part protein has been optionally post-translationally modified, in particular glycosylated, phosphorylated, amidated, methylated, acetylated, ADP-ribosylated, hydroxylated, provided with a membrane anchor, cleaved or shortened,

e. an antibody, preferably a monoclonal or polyclonal antibody, against one of the proteins or part proteins according to point d),

f. a cell, preferably an amphibia cell, bacteria cell, yeast cell, insect cell or an immortalized or native mammalian cell, containing a polynucleotide according to one of points a) or b), a vector according to point c), a protein or part protein according to point d) or an antibody according to point e)

g. a compound according to one of claims 12 or 13 and/or

h. an active compound, preferably a low molecular weight active compound, which binds to a protein or part protein according to point a),

for the preparation of a diagnostic agent for diagnosis of a pain state. Diagnostics is understood here as meaning the analysis of symptoms assigned to a disease syndrome, and investigations of activity are understood as meaning investigations of the activity of substances to be tested, in particular their medicinal activity.

The invention furthermore also provides a process for the preparation of a peptide or protein according to the invention, in which a cell according to the invention which contains a polynucleotide according to the invention and/or a vector according to the invention is cultured and the peptide or protein is optionally isolated.

The invention also provides the use of

a. a polynucleotide, preferably a DNA or RNA, which codes for BNPI or DNPI or a polynucleotide, preferably a DNA or RNA, which corresponds to the extent of at least 90%, preferably 95%, in particular to the extent of at least 97%, to one of the nucleotide sequences shown in FIGS. 1a), 1c), 1e), 1g), 2a), 2c) or 2e) (SEQ ID NOS 1, 3, 5, 7, 9, 11 or 13),

b. a polynucleotide, in particular an antisense polynucleotide or a PNA, preferably a DNA enzyme or ribozyme, a ribozyme or other DNA enzyme or a catalytic RNA or DNA, which has a nucleotide sequence which is capable of binding specifically to one of the polynucleotides listed under point a),

c. a vector containing a polynucleotide according to one of points a) or b), in particular an expression vector and/or in particular derived from a virus, for example the adenovirus, adeno-associated virus or herpes virus, and/or in particular containing at least one LTR, poly A, promoter and/or ORI sequence,

d. BNPI or DNPI and/or a protein according to one of FIGS. 1b), 1d), 1f), 1h), 2b), 2d) or 2f) (SEQ ID NOS 2, 4, 6, 8, 10, 12 or 14) and/or a protein which is similar to one of these abovementioned proteins to the extent of at least 90% and/or a protein for which a polynucleotide according to one of FIGS. 1a), 1c), 1e), 1g), 2a), 2c) or 2e) (SEQ ID NOS 1, 3, 5, 7, 9, 11 or 13) or a polynucleotide which is similar thereto to the extent of at least 90% codes, and/or a protein which is coded by a nucleic acid which binds under stringent conditions to a polynucleotide according to one of FIGS. 1a), 1c), 1e), 1g), 2a), 2c) or 2e) (SEQ ID NOS 1, 3, 5, 7, 9, 11 or 13) or antisense polynucleotides thereof or a part protein of one of the abovementioned proteins which is at least 10, preferably at least 15, in particular at least 20 amino acids long, wherein the protein or part protein has been optionally post-translationally modified, in particular glycosylated, phosphorylated, amidated, methylated, acetylated, ADP-ribosylated, hydroxylated, provided with a membrane anchor, cleaved or shortened,

e. an antibody, preferably a monoclonal or polyclonal antibody, against one of the proteins or part proteins according to point d),

f. a cell, preferably an amphibia cell, bacteria cell, yeast cell, insect cell or an immortalized or native mammalian cell, containing a polynucleotide according to one of points a) or b), a vector according to point c), a protein or part protein according to point d) or an antibody according to point e)

in a method for detecting pain-regulating substances.

Generally, it is preferable for all the abovementioned uses according to the invention for the pain to be chosen from

chronic pain, in particular musculoskeletal pain; neuropathic pain, in particular allodynic pain, mechanical hyperalgesia or diabetic neuropathy; visceral pain, cerebral pain, peripheral pain or inflammation-related pain, in particular peripheral inflammation pain; and migraine, cluster headache or pain with trigeminus neuralgia.

The polynucleotide used according to the invention also includes the gene fragments described themselves, as well as a polynucleotide which corresponds either completely or at least in parts to the coding sequence of the gene corresponding to the fragment. This also means polynucleotides which have at least 90%, preferably 95%, in particular at least 97% agreement in the base sequence with the coding sequence of the polynucleotides shown or the coding sequence of the gene. It is furthermore preferable for the polynucleotide to be RNA or single- or double-stranded DNA, in particular mRNA or cDNA. It is also preferable for the polynucleotide to be an antisense polynucleotide or PNA which has a sequence which is capable of binding specifically to a polynucleotide according to the invention. PNA is understood here as meaning “peptidic nucleic acid”, which indeed carries the base pairs but the backbone of which is bound peptidically. An antisense polynucleotide shows the complementary base sequence to at least a part of a base nucleic acid. It is also preferable for the polynucleotide to be part of a ribozyme or other DNA enzyme or of a catalytic RNA or DNA. Ribozyme is to be understood as meaning a catalytically active ribonucleic acid, and DNA enzyme is to be understood as meaning a corresponding deoxyribonucleic acid, that is to say catalytic RNA or DNA.

The vector used according to the invention is understood as meaning a nucleic acid molecule which serves to contain or transfer foreign genes in manipulation by genetic engineering. It is particularly preferable here for the vector to be an expression vector. It therefore serves for expression of the foreign gene contained therein, the polynucleotide. Such a vector which is derived from a virus, for example the adenovirus, adeno-associated virus or herpes virus, and/or it contains at least one LTR, poly A, promoter and/or ORI sequence is furthermore preferred. An LTR is a “long terminal repeat”, a section at the end, for example in viruses. Poly A sequence is a tail more than 20 adenosine radicals long. A promoter sequence is the control region for the transcription.

For a protein used or a part protein derived therefrom, it is preferable for this to have been post-translationally modified, for it to have been, in particular, glycosylated, phosphorylated, amidated, methylated, acetylated, ADP-ribosylated, hydroxylated, provided with a membrane anchor, cleaved or shortened. Post-translational modifications can be found, for example, in Voet/Voet, Biochemistry, 1st Edition, 1990, p. 935-938.

For a use according to the invention, it is particularly preferable here for the polynucleotide (optionally according to point a) and/or point b)) to be an RNA or a single- or double-stranded DNA, in particular, mRNA or cDNA.

For a use according to the invention, it is particularly preferable here for the polynucleotide (optionally according to point b)) to be part of a ribozyme or other DNA enzyme or of a catalytic RNA or DNA.

For a use according to the invention, it is particularly preferable here for the vector (optionally according to point c)) to be an expression vector.

For a use according to the invention, it is furthermore particularly preferable here for the vector (optionally according to point c)) to be derived from a virus, for example the adenovirus, adeno-associated virus or herpes virus, and/or to contain at least one LTR, poly A, promoter and/or ORI sequence.

For a use according to the invention (not gene therapy), it is particularly preferable here for the protein or part protein (optionally according to point d)) to have been post-translationally modified, in particular to have been glycosylated, phosphorylated, amidated, methylated, acetylated, ADP-ribosylated, hydroxylated, provided with a membrane anchor, cleaved or shortened.

For a use according to the invention (not gene therapy), it is particularly preferable here for the antibody (optionally according to point e)) to be a monoclonal or polyclonal antibody.

For a use according to the invention, it is particularly preferable here for the cell (optionally according to point f)) to be an amphibia cell, bacteria cell, yeast cell, insect cell or an immortalized or native mammalian cell.

For a use according to the invention, it is particularly preferable here for the compound (optionally according to point g)) to be a low molecular weight compound.

For a use according to the invention, it is particularly preferable here for the active compound mentioned, according to point h), to be a low molecular weight active compound.

The invention also provides a process for pain treatment of a non-human mammal or human which or who requires treatment of pain, in particular chronic pain, by administration of a pharmaceutical formulation according to the invention, in particular one comprising a substance according to the invention and/or an active compound which binds BNPI and/or DNPI.

The administration can take place, for example, in the form of a pharmaceutical formulation as described above.

Overall, an important basis of the invention is the identification of pain-regulated genes and gene fragments. The screening method is based on this. However, the use for diagnosis or therapy is also available, as already stated. Appropriate possible uses and further embodiment examples are explained in the following.

1. Therapy of Chronic Pain

mRNA expression of kinases was investigated by in situ hybridization in spinal cord tissue. In the spinal cord, the primary sensory neurons project to subsequent central nervous neurons, these being, in addition to supraspinal processes, the central switching site for nociceptive information. Numerous experiments have shown that the development of chronic states of pain is based on plastic changes in the nervous system (as an overview see Corderre et al., 1993; Zimmermann and Herdegen, 1996). In the neurons of the dorsal root ganglia and spinal cord in particular, plastic changes which are accompanied by regulation of pain-relevant genes have been described. Gene regulation in the spinal cord has thus been described for a number of neurotransmitter receptors which are of importance for pain therapy (see table 1). On this basis, the cDNA sequences found which are regulated under pain could be used for therapy (gene therapy, antisense, ribozymes) and diagnosis of chronic states of pain.

1.1 Antisense Strategies

Constructs which are derived from the nucleic acid sequence of the complete cDNA or from part regions and which can reduce the mRNA or protein concentration are established here. These can be e.g. antisense oligonucleotides (DNA or RNA), which have an increased stability towards nucleases, possibly using modified nucleotide units (e.g. O-allyl-ribose). Furthermore, the use of ribozymes, which, as enzymatically active RNA molecules, catalyse a specific cleavage of the RNA, is conceivable. In addition, vectors which express the sequences according to the invention or part regions of these nucleotide sequences under control of a suitable promoter and are therefore suitable for an in vivo or ex vivo therapy could also be employed. Antisense constructs which, under exchange of the phosphate backbone of nucleotide sequences (e.g. PNAs, i.e. peptide nucleic acid) or by using non-traditional bases, such as inosines, queosines or wybutosines, as well as acetyl-, methyl-, thio- and similarly modified forms of adenine, cytidine, guanosine u, thymidine and uridine, cannot be degraded or can be degraded to a relatively low degree by endogenous nucleases are additionally also possible.

1.2. Antagonists/Agonists or Inhibitors/Activators of the Gene Products According to the Invention used in the Screening Method.

This includes substances which, by binding to the gene product, modify the function thereof. These can be:

1.2.1. Organic chemical molecules which are found in the context of an active compound screening using the gene products of the cDNA according to the invention as binding partners.

1.2.2. Antibodies, whether polyclonal, chimeric, single-chain, F_{ab} fragments or fragments from phage banks, which preferably specifically influence the function as neutralizing antibodies via binding to the gene products.

1.2.3. Aptamers, i.e. nucleic acids or nucleic acid derivatives with protein-binding properties. These also include so-called mirror-mers, which are mirror-image and therefore stable oligonucleotides obtained by mirror evolution and can bind a target molecule with a high affinity and high specificity (Kluβmann et al., 1996).

1.3. Gene Therapy

The sequences described can be employed for therapy of neurological diseases, in particular chronic states of pain, by using them, after cloning into suitable vectors (e.g. adenovirus vectors or adeno-associated virus vectors), for in vivo or ex vivo therapy in order there e.g. to counteract an over-expression or under-expression of the endogenous gene product, to correct the sequence of the defective gene product (e.g. by trans-splicing with the exogenous construct) or to provide a functional gene product.

2. Diagnosis

Polynucleotide sequences (oligonucleotides, antisense DNA & RNA molecules, PNAs) which are derived from the nucleotide sequences used in the screening method etc. could be employed for diagnosis of states or diseases associated with an expression of these gene sequences. Examples of these states or diseases include neurological diseases, including chronic pain or neuropathic pain (caused e.g. by diabetes, cancer or AIDS), or neurodegenerative diseases, such as Alzheimer's, Parkinson's, Huntington's Chorea, Jacob-Creutzfeld's, amyotrophic lateral sclerosis and dementias. The nucleotide sequences can serve in diverse ways (northern blot, southern blot, FISH analysis, PRINS analysis, PCR) either for identification of the gene product or deviating diagnostically relevant gene products or for quantification of the gene product. In addition to nucleic acid diagnostics, antibodies or aptamers against the protein coded by the nucleic acids according to the invention can also be employed for diagnostics (e.g. by means of ELISA, RIA, immunocytochemical or immunohistochemical methods) in order to identify the protein or deviating forms and to quantify the protein.

In respect of gene diagnostics, nucleic acid probes derived from the nucleotide sequences according to the invention could be employed for determination of the gene locus (e.g. by FISH, FACS, artificial chromosomes, such as YACs, BACs or P1 constructs).

Certain embodiments of the present invention may be understood more readily by reference to the figures and specific examples. The following examples, figures and the terminology used herein are for the purpose of describing particular embodiments and are intended to illustrate the invention without limiting it thereto.

### BRIEF DESCRIPTION OF THE FIGURES

FIG. 1a is a cDNA sequence (SEQ ID NO: 1) of BNPI, human; AN: NM_{—}020309.

FIG. 1b is an amino acid sequence (SEQ ID NO: 2) of PIM1-kinase, human; AN: NM_{—}020309.

FIG. 1c is a cDNA sequence (SEQ ID NO: 3) of BNPI, human; no.: AAT42064 from WO96/34288.

FIG. 1d is an amino acid sequence (SEQ ID NO: 4) of BNPI, human; no.: AAT42064 from WO96/34288.

FIG. 1e is a cDNA sequence (SEQ ID NO: 5) of BNPI, rat; AN: U07609.

FIG. 1f is an amino acid sequence (SEQ ID NO: 6) of BNPI, rat; AN: U07609.

FIG. 1g is a cDNA sequence (SEQ ID NO: 7) of BNPI, mouse; AN: XM_{—}133432.

FIG. 1h is an amino acid sequence (SEQ ID NO: 8) of BNPI, mouse; AN: XM_{—}133432.

FIG. 2a is a cDNA sequence (SEQ ID NO: 9) of DNPI, human; AN: AB032435.

FIG. 2b is an amino acid sequence (SEQ ID NO: 10) of DNPI, human; AN: AB032435.

FIG. 2c is a cDNA sequence (SEQ ID NO: 11) of DNPI, rat; AN: AF271235.

FIG. 2d is an amino acid sequence (SEQ ID NO: 12) of DNPI, rat; AN: AF271235.

FIG. 2e is a cDNA sequence (SEQ ID NO: 13) of DNPI, mouse; AN: NM_{—}080853.

FIG. 2f is an amino acid sequence (SEQ ID NO: 14) of DNPI, mouse; AN: NM_{—}080853.

FIG. 3 is a separation of radioactively labeled RFDD-PCR fragments in a 6% denaturing PAA gel (see example 1).

FIG. 4 is an upwards regulation of DNPI and BNPI protein expression in primary sensory rat DRG neurons and fibers after collagen-induced arthritis. (see example 2).

FIG. 5 is a differential expression of DNPI and BNPI in synapses of pain conduction and motor areas of the lumbar spinal cord of the rat (see example 3a).

FIG. 6 is a differential expression of DNPI and BNPI in synapses of the dorsal horn pain conduction areas of the lumbar spinal cord of the rat (see example 3b).

FIG. 7 is a differential expression of DNPI and BNPI in synapses of pain conduction of the sacral spinal cord of the rat (see example 3c).

FIG. 8 is a differential expression of DNPI and BNPI in synapses of medullo-cervicospinal pain conduction of the trigeminal nerve of the rat (see example 3d).

FIG. 9 is a differential expression of DNPI and BNPI in pain-relevant brain regions of the rat (see example 3e).

FIG. 10 is a differential expression of DNPI and BNPI in pain-relevant brain regions of the rat (see example 3f).

FIG. 11 is a differential expression of DNPI and BNPI in pain-relevant brain regions of the rat (see example 3g).

FIG. 12 is a differential expression of DNPI and BNPI in pain-relevant brain regions of the rat (see example 3h).

### EXAMPLES

### Example 1

### Identification of Pain-Regulated Genes by Means of RFDD-PCR

A) Procedure

The following procedure was chosen:

CFA-induced arthritis in the rat in which complete Freund's adjuvant is injected into the tail root was chosen as the starting point for isolation of pain-regulated genes. The target tissue in which the pain-regulated expression of the genes according to the invention was detected was the dorsal root ganglia of the fifth lumbar segment. Four methods are available for isolation of differentially regulated genes:

cDNA-RDA (cDNA-representational difference analysis; Hubank & Schatz, 1994)

DDRT-PCR (differential display RT-PCR; Liang & Pardee 1992, Bauer et al., 1994), There have since been improved modifications of this, such as the so-called “restriction fragment differential display PCR” (RFDD-PCR), which allows a more reproducible reaction by additional restriction fragmenting of the cDNA in combination with an optimized PCR amplification and furthermore detects fragments to an increased extent in the coding region (Ivanova et al., 1995).

Subtractive hybridization (Watson & Margulies, 1993)

SAGE (serial analysis of gene expression, Velculescu et al., 1995).

A comparative evaluation of the methods mentioned led to selection of RFDD-PCR, since in contrast to subtractive hybridization and SAGE, this method is capable of detecting both upwards- and downwards-regulated genes and also rare transcripts and moreover provides an abundance of results within short periods of time.

B) Material and Methods

Isolation and Characterization of Pain-Regulated cDNA Sequences Animal Model: CFA-Induced Polyarthritis

Adjuvant arthritis (AA) is an induced form of (sub)chronic arthritis. It is induced by immunizing rats with a suspension of mycobacteria in oil. The disease thereby induced is an autoimmune arthritis which is mediated by T cells and which—since, however, no defined autoantigen is employed during the induction—corresponds to an arthritis which occurs spontaneously in humans. AA is often used for investigations of immunological aspects of rheumatoid arthritis. Furthermore, the model is used for testing antiinflammatory and analgesic substances. AA is a fairly aggressive form of arthritis. The inflammation process of AA is indeed self-healing, but severe joint changes nevertheless persist. The severity of the disease can be quantified by drawing up an arthritis index. All four paws are inspected here for redness, swelling and deformation of the joints. The course of the disease can furthermore be characterized more closely via determination of the body weight and of the paw swelling by means of plethysmography and by histological examinations of the joints.

The arthritis is induced by intracutaneous injection of CFA (100 μl of the 5 mg/ml stock solution) into the tail root (dorsal). The severity of the arthritis is determined with the aid of a scoring index by daily observation of the animals for mobility, reddening of the skin and swellings of the tarsal and carpal joint. The onset of visible inflammations of the tarsal or carpal joint starts on about day 10 after immunization. The severity of the disease increases over a period of 10-14 days, reaches an optimum which is maintained for about 6-7 days, to then subside again. If rats were immunized only with IFA, no arthritis was induced.

Removal of Tissue.

The animal are decapitated and the dorsal root ganglia are removed after lubalectomy and immediately frozen in liquid nitrogen.

RNA Isolation.

The total RNA was isolated from the tissue samples with the Trizol Kit (Life Technologies) in accordance with the manufacturer's instructions. The RNA was quantified by UV spectrometry (extinction at 260 nm) and checked for integrity by denaturing gel electrophoresis in a formaldehyde-agarose gel (Sambrook et al., 1989).

DNase Digestion.

Before use in the DDRT-PCR, any traces of genomic DNA are removed by DNase digestion. In this, in each case 6 μg RNA were incubated in a total volume of 100 μl in 1× First-Strand Buffer (Life Techn.) and 10 units of RNase-free DNasel (Boehringer Mannheim) for 15 minutes at 37° c. After phenol/chloroform extraction, the RNA was precipitated by addition of 1/10 vol. sodium acetate pH 5.2 and 2.5 vol. ethanol, dissolved in DEPC water, quantified by UV spectrometry and characterized by renewed formaldehyde-agarose gel electrophoresis.

Reverse Transcription.

In each case 1 μg of DNasel-digested RNA were subjected to reverse transcription with the aid of the displayProfile Kit (Display Systems Biotech, Vista, USA) in accordance with the manufacturer's instructions and double-stranded cDNA was produced. After purification of the cDNA by phenol/chloroform extraction and ethanol precipitation, the efficiency of the cDNA synthesis was detected by gel electrophoresis in a 1.5% agarose gel.

Taq1 Restriction Digestion.

In each case 10 μl of the double-stranded cDNA were digested with the restriction enzyme Taq1. This was also carried out with the aid of the displayProfile Kit (Display Systems Biotech, Vista, USA) in accordance with the manufacturer's instructions. Starting from this batch, adapters are ligated to the digested cDNA.

³³P End Labelling Reaction.

For subsequent detection of the fragments, one of the two primers (so-called O-extension primers) was radioactively labeled by an end labeling reaction with T4 polynucleotide kinase and [γ³³P]ATP. This was also carried out with the aid of the displayProfile Kit (Display Systems Biotech, Vista, USA) in accordance with the manufacturer's instructions.

PCR Amplification of the cDNAs.

After ligation, in each case 0.2 μl of the cDNA are amplified in parallel reaction batches with the labeled o-extension primer and one of the 64 Eu primers and the reaction batches are separated by electrophoresis in a 6% Tris-taurine-EDTA-polyacrylamide gel. The gel was then dried for one hour at 80° C. and exposed overnight on a BASIII detection screen (Fuji). The STORM-Phosphorus Imager (Molecular Dynamics) using the ImageQuant software was used for the evaluation. The autoradiography data were printed on film in the same scale, which was then used for cutting out the fragments.

Reamplification of the DDRT-PCR Fragments.

Differentially regulated PCR bands were cut out of the gel with a scalpel and eluted from the piece of gel by boiling for 15 minutes in 50 μl Tris-EDTA buffer, were reamplified by PCR with the aid of the displayProfile Kit (Display Systems Biotech, Vista, USA) in accordance with the manufacturer's instructions. The temperature profile corresponded to the original PCR reaction (see above). 10 μl sample buffer (0.25% bromophenol blue, 0.25% xylenecyanol FF, 30% glycerol) then added to the PCR batches, separation carried out by gel electrophoresis in a 3% TAE-agarose gel with 10 μg/ml ethidium bromide and PCR products of the expected size cut out of the gel.

Cloning into TA Cloning Vectors.

The fragments cut out were purified with the Qiaquick Gel Extraction Kit (Qiagen) in accordance with the manufacturer's instructions, concentrated to dryness and taken up in 5 μl doubly dist. water. They were then ligated into the pCRII-TOPO vector by means of the TOPO TA Cloning Kit (Invitrogen) in accordance with the manufacturer's instructions and transformed in TOP10 F′-E. coli cells. The transformation batch was plated out on LB-agar plates with 100 μg/ml ampicillin, which had been treated beforehand with 50 μl 2% ×-Gal (Sigma) and 50 μl isopropyl thiogalactoside (Sigma). The white bacteria clones obtained after incubation for 15 hours at 37° C. were transferred into 5 ml LB liquid medium with 100 μg/ml ampicillin (100 μg/ml) and incubated overnight at 37° C., while shaking. Plasmid DNA was isolated from these cultures using the Qiagen Spin Miniprep Kit (Qiagen) in accordance with the manufacturer's instructions and in each case 5 μl of the plasmid DNA were characterized by EcoRI restriction digestion and subsequent TAE-agarose gel electrophoresis.

Sequence Analysis.

In this, in each case 500 ng of the plasmid DNA were sequenced with the T7-PCR primer using the Dye Terminator Cycle Sequencing Kit (Perkin-Elmer) in accordance with the manufacturer's instructions and the reactions were analyzed by means of the automatic sequencer ABI 370 (Applied Biosystems Inc.). The DNA sequences were compared with the gene libraries using bioSCOUT software (LION, Heidelberg).

C) Result

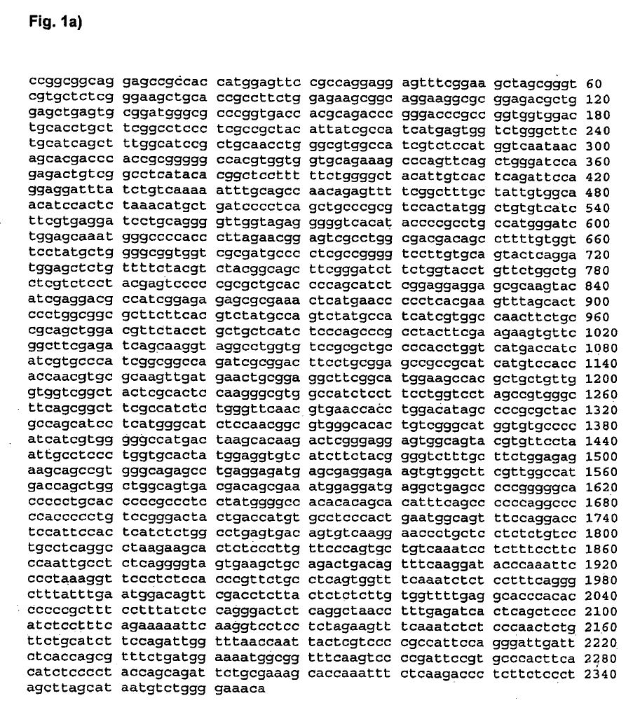

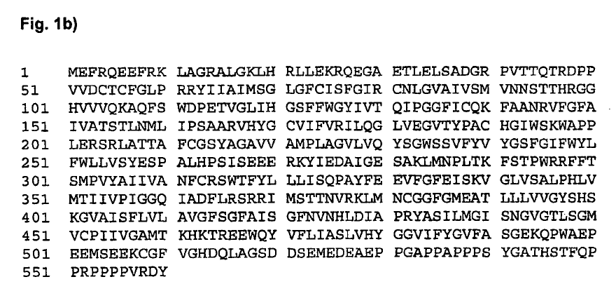

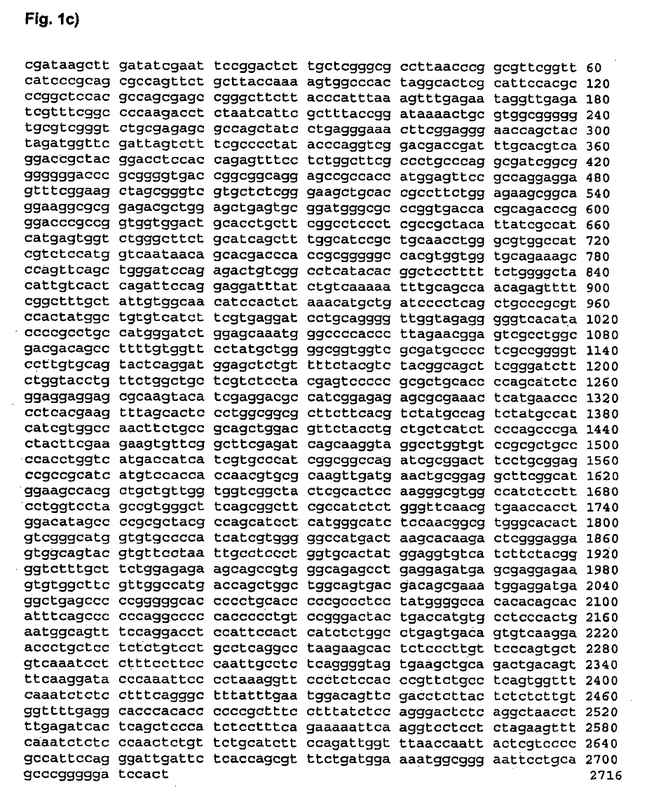

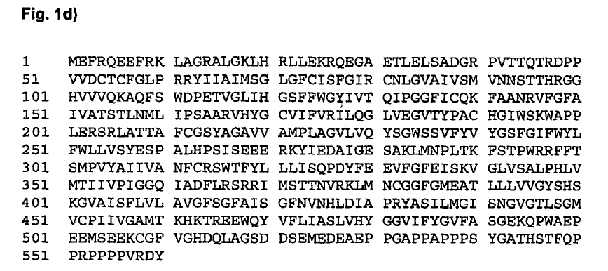

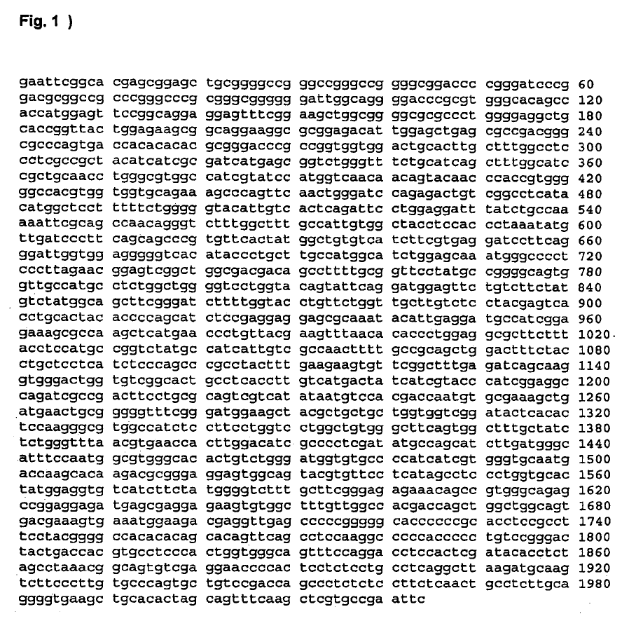

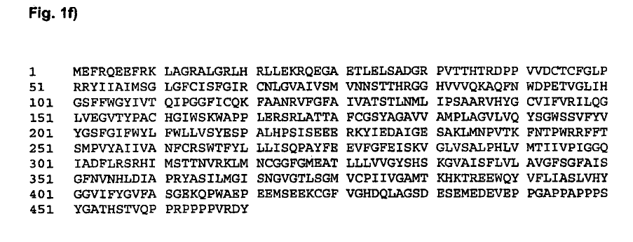

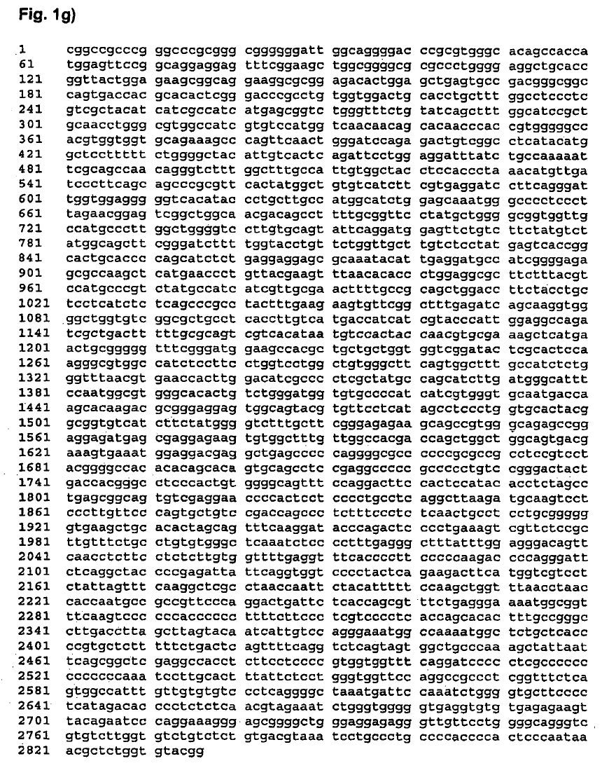

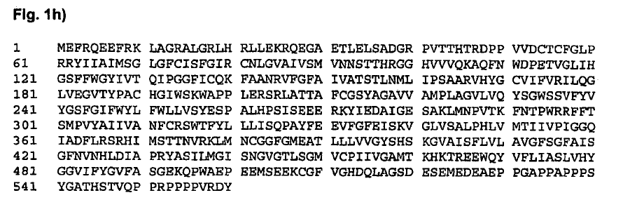

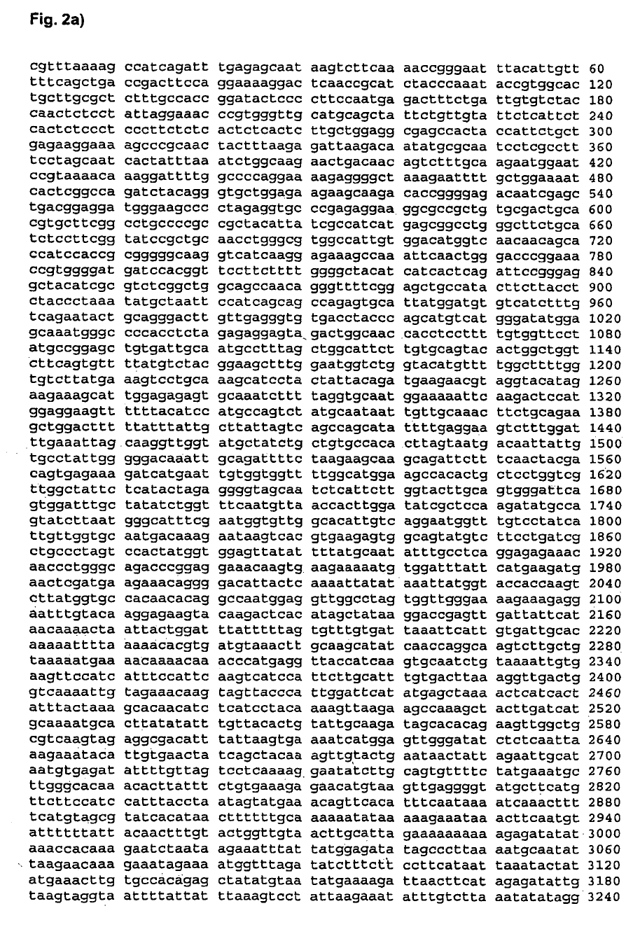

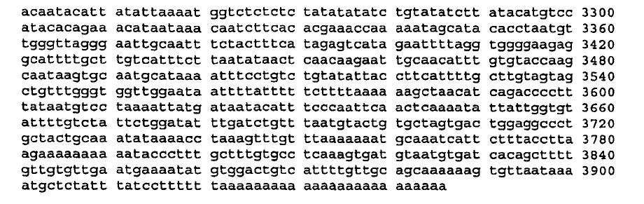

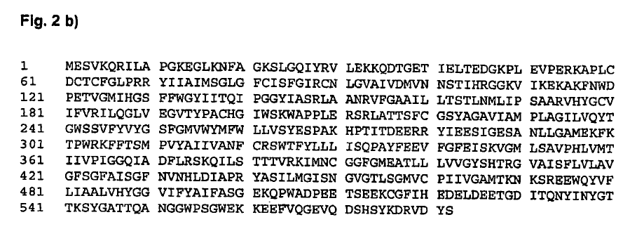

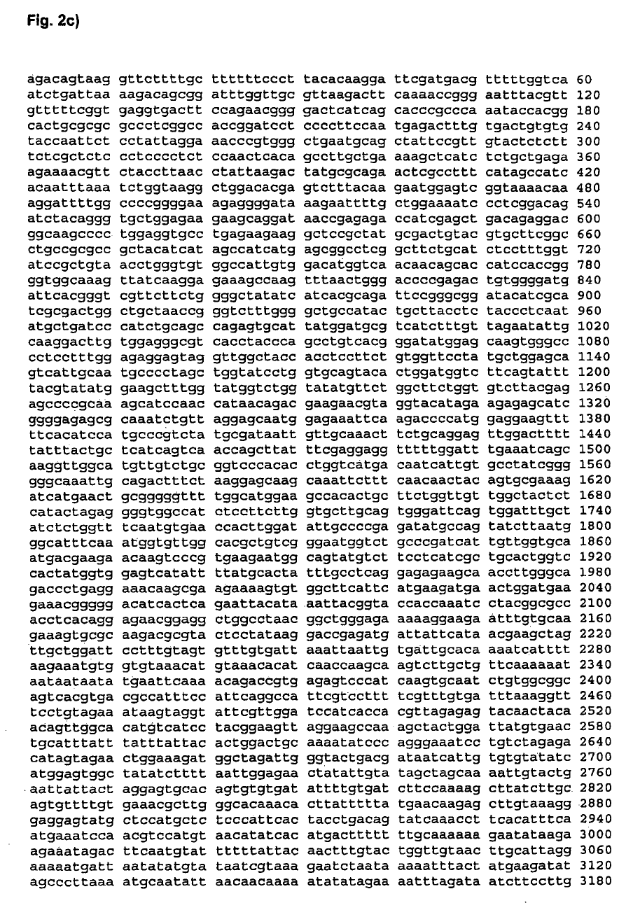

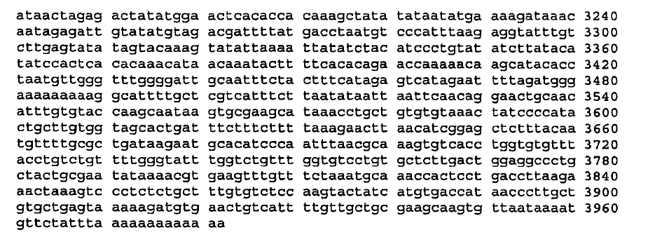

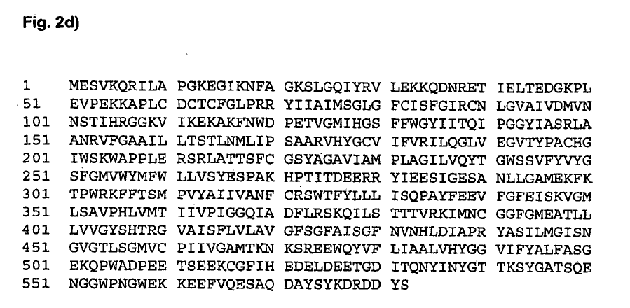

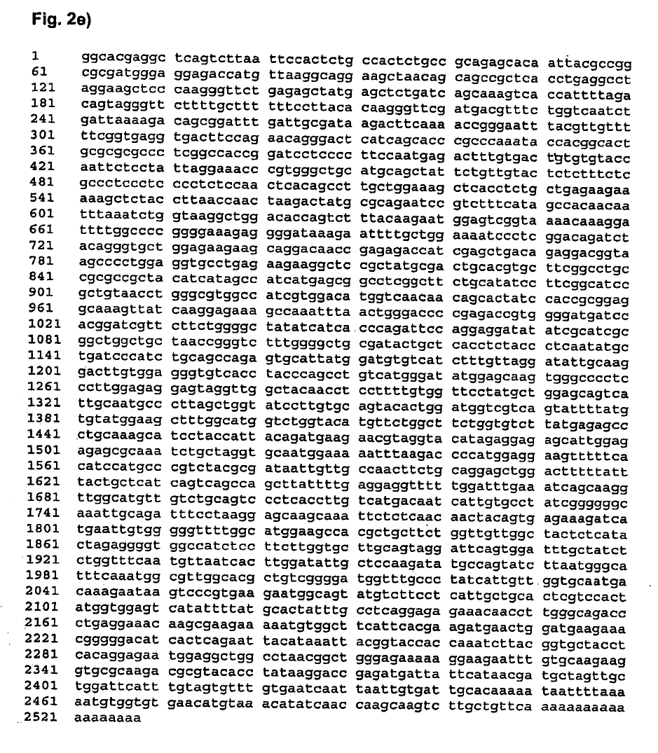

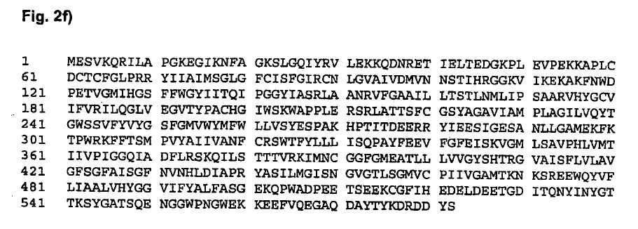

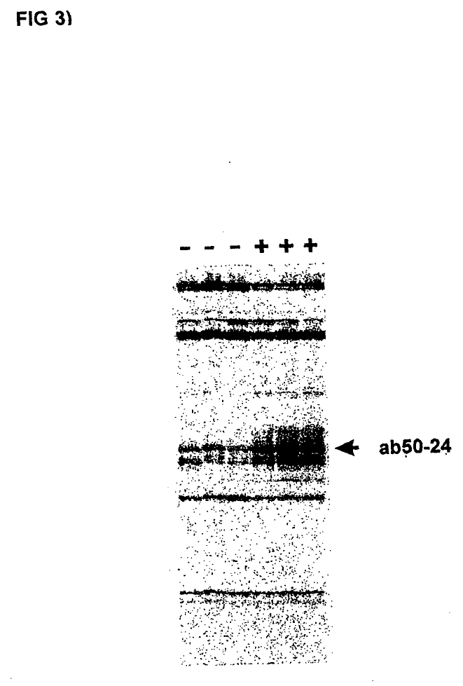

A corresponding autoradiogram is shown in FIG. 3. The autoradiogram shows the separation of PCR fragments which have formed by amplification of various cDNAs. The cDNAs were synthesized by reverse transcription from total RNA from L5 spinal ganglia. The total RNA was isolated from control animals (−) and CFA-treated animals (+). The fragment ab50-24 which, as shown by means of the RFDD method, has an upwards regulation is identified with an arrow. The fragment ab50-24 shows a highly significant homology to the cDNA sequence AC no. AAT42064 of hBNPI (see FIG. 1c). It is therefore demonstrated that BNPI is expressed more intensively under the conditions of a CFA treatment.

### Example 2

### Identification of Pain-Regulated Genes via Immunocytochemical Staining

The following procedure was chosen.

The so-called CIA model(collagen-induced arthritis) in the rat, in which colagen is injected in order to induce arthritis in the rat was chosen as the starting point for isolation of pain-regulated genes.

The procedure corresponded to the method described by Persson S., Schäfer MK-H., Nohr D., Ekström G., Post C., Nyberg F. and Weihe E. (1994), Neuroscience 63; 313-326 and Nohr D., Schäfer MK-H., Romeo H., Persson S., Nyberg F. Post C. and Weihe E. (1999), Neuroscience 93; 759-773, the disclosure of this article expressly being made part of the disclosure of the invention submitted here.

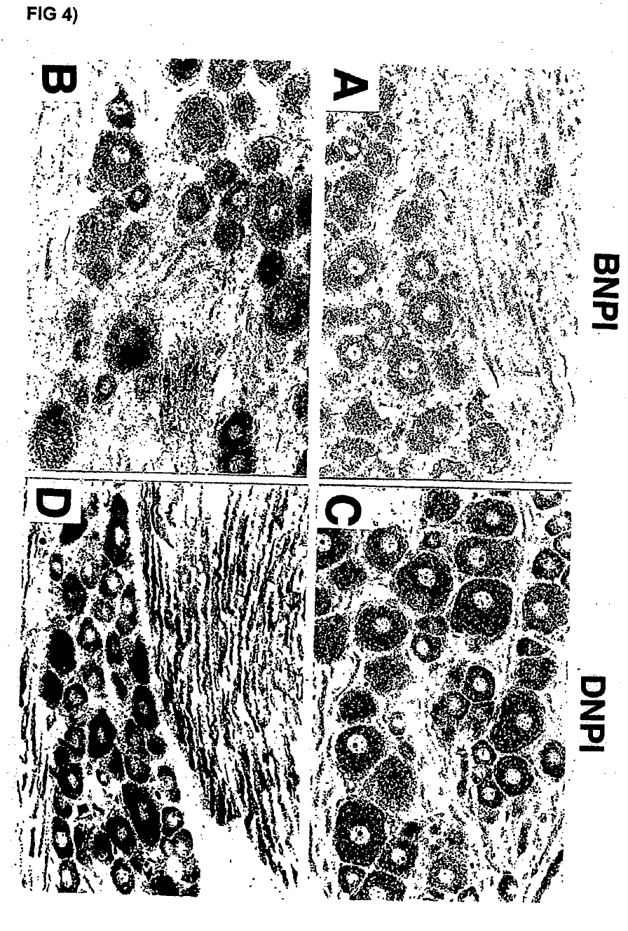

Polyclonal rabbit antisera against the recombinant DNPI or BNPI fusion protein were used for the immunohistochemical staining. It was found in FIG. 4 that the intensity of the DNPI and BNPI immunostaining in the lumbar dorsal root ganglion of the arthritic rat (B and D/CIA) increased compared with the control animals (A and C/CTLR). The increase both in the cell bodies and the fiber staining in B compared with A and in C compared with D is to be noted.

### Example 3

### Differential Consideration of the Expression Between DNPI and BNPI via Immunocytochemical Staining

Polyclonal rabbit antisera against the recombinant DNPI and BNPI fusion protein were used for the immunohistochemical staining. Generally, sections of various regions of the CNS were prepared and the expression of DNPI was compared with that of BNPI.

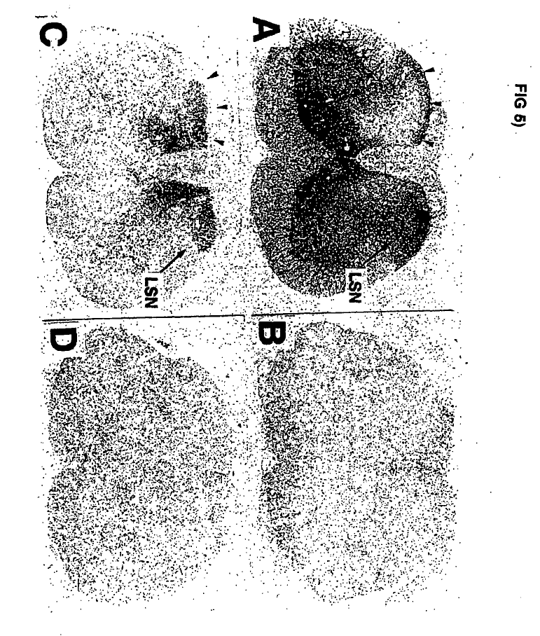

### Example 3a on FIG. 5)

The differential distribution of the immune reactivity of BNPI and DNPI in the lumbar spinal cord of the rat is to be seen. The adjacent deparafinized sections A- to D are stained as follows:

A=anti-DNPI;

B=anti-DNPI preadsorbed with DNPI fusion protein;

C=anti-BNPI;

D=anti-BNPI preadsorbed with BNPI fusion protein;

The DNPI (A) and BNPI (C) immunodyestuffs were completely preadsorbable with homologous recombinant BNPI (D) and BNPI (B) fusion protein, which proves the specificity of the immune reaction.

The mutually exclusive distribution pattern of DNPI and BNPI immunostaining in the outer and deep dorsal horn is remarkable. (A;C). Pointwise immunostaining of DNPI is in the synaptic endings of the outer dorsal horn (lamina 1 and substantia gelatinosaa) (arrow in A), while BNPI immune reactivity is completely absent (arrows in B). Accumulation of intense positive pointwise BNPI immunostaining exists in the deeper dorsal horn, while DNPI staining is relatively low. DNPI is present in the lateral spinal nucleus (LSN in A), while BNPI is completely absent (LSN in C). DNPI is abundant in the lamina X around the central canal, while BNPI is rare. BNPI immunostaining is weak in the lateral ventral horn and slight or absent in the medial ventral horn. Pointwise DNPI staining is abundant through the entire ventral horn, but somewhat less in the lateral horn compared with the medial ventral horn. There is a weak BNPI and DNPI staining in some cell bodies of the ventral horn motoneurone, but this was not preadsorbed by the homologous transport fusion proteins and was therefore classified as non-specific.

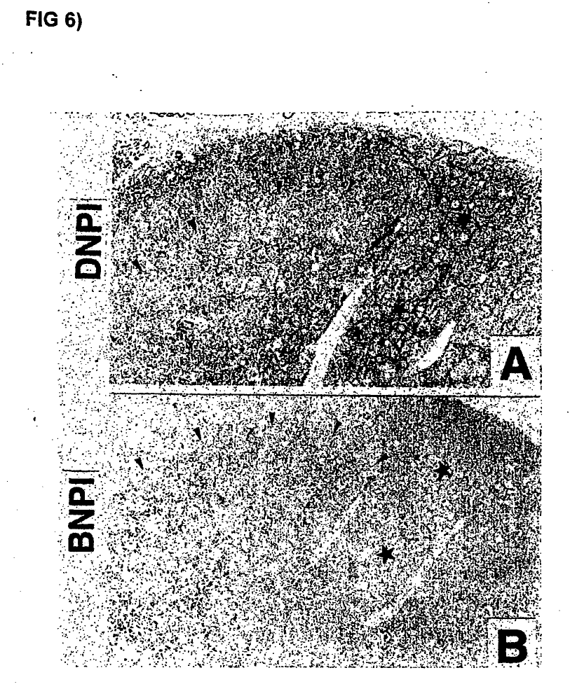

### Example 3b on FIG. 6)

The differential distribution of the immune reactivity of BNPI and DNPI in the left lateral superficial dorsal lumbar spinal cord of the rat is to be seen. A and B, stained in each case for BNPI (A) and DNPI (B), show many pointwise stains for DNPI, which are concentrated in the lamina I and substantia gelatinosa, where BNPI is almost completely absent. Dense complexes of DNPI-positive points are furthermore to be seen in the lateral spinal nucleus, where BNPI is almost completely absent. Fine DNPI-positive points are also to be found in the deeper dorsal horn, although in a lower density.

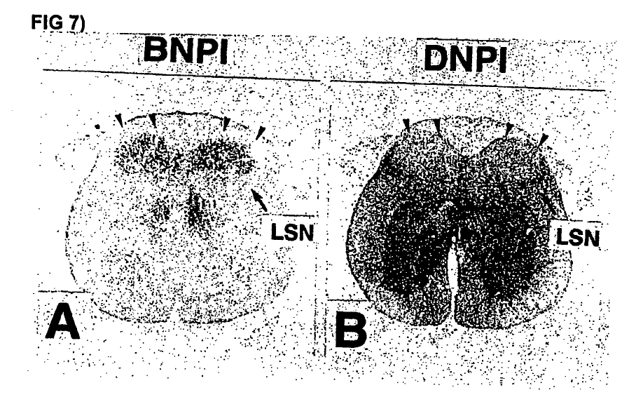

### Example 3c on FIG. 7)

The differential distribution of the immune reactivity of BNPI and DNPI in the sacral spinal cord of the rat is to be seen. The adjacent sections A and B, stained in each case for BNPI (A) and DNPI (B), show mutual exclusion zones of pointwise DNPI and BNPI immunostaining in the dorsal horn. DNPI is present in the entire grey matter and is concentrated in the very outer layers of the dorsal horn, where a narrow band forms at the boundary to the white matter. DNPI is abundant in the lateral spinal nucleus and in the lamina X, and also in the lamina V/VI and in the entire ventral horn. BNPI is abundant in the deep dorsal horn and rare in the ventral horn.

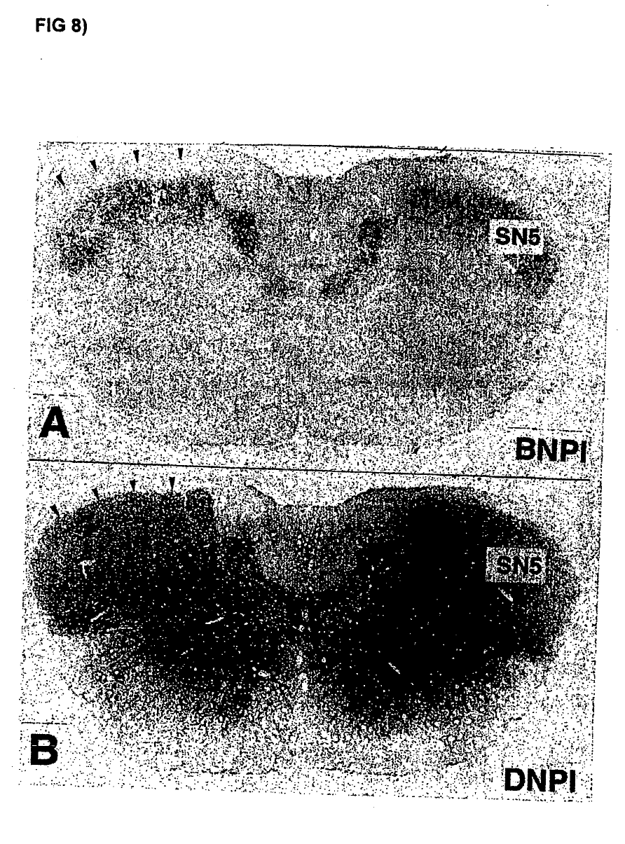

### Example 3d on FIG. 8)

The differential distribution of the immune reactivity of BNPI and DNPI in the lower medulla oblongate at the transition to the cervical spinal cord can be seen. The adjacent sections A and B, in each case stained for BNPI (A) and DNPI (B), show a preferred accumulation of the BNPI staining in the medial part of the spinal trigeminal nucleus and in the middle and lower part of the dorsal medulla. only a very weak staining is to be seen with BNPI in the ventral medulla. DNPI is abundant in the grey matter of the medulla. DNPI staining overlaps with the BNPI staining in the inner spinal nucleus V. It is to be noted that BNPI is also to be found in the upper spinal trigeminal nucleus, which is the same as the spinal substantia geloatinosa. DNPI staining is weaker in areas in which BNPI is present, weaker than in areas where BNPI is low or absent. A few BNPI points are to be seen in the ventral grey motor area.

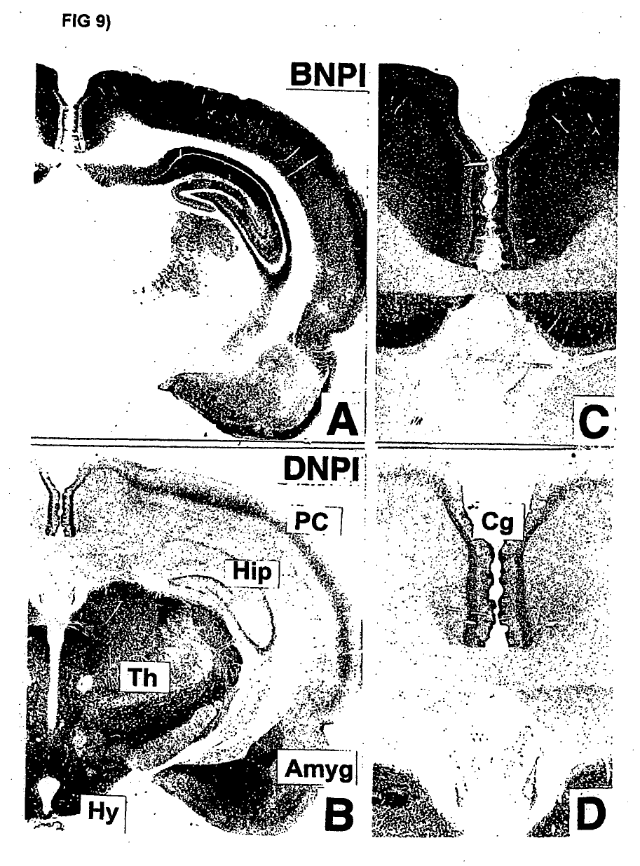

### Example 3e on FIG. 9)

Complementarily differential distribution of DNPI and BNPI immune reactivity in 2 consecutive sections of the rat brain in pain-relevant brain regions, such as the sensory parietal cortex; cingular cortex, thalamus, corpus amygdaloideum and also hypothalamus. DNPI is concentrated in the cortex in the granular sensory layers, in particular in lamina IV; BNPI is abundant in the cortex but weaker in lamina IV than in other laminae. In the cingular cortex (C vs D as a magnification), the distribution of DNPI and BNPI is complementarily mutually exclusive or reciprocal in the density of the particular synapses. DNPI clearly predominates over BNPI in the thalamus, BNPI is sparse in the hypothalamus, DNPI abundant. Abundant BNPI predominates in the hypocampus over sparse DNPI with mutually complementary distribution.

Thalamus=Th,

Amygdala=Amyg.

Hippocampus=Hip,

Cingular cortex=Cg,

Hypothalamus=Hy,

Parietal cortex=PC.

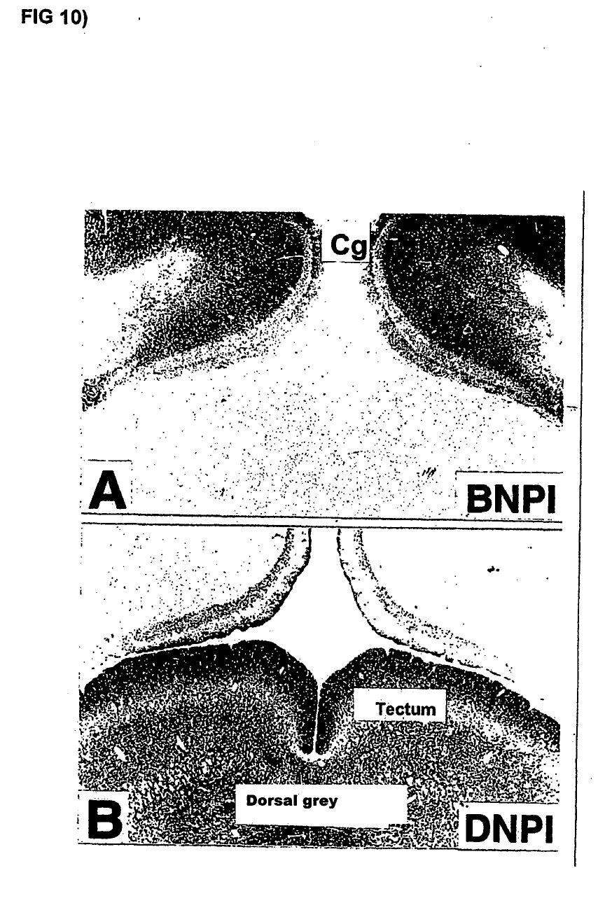

### Example 3f on FIG. 10)

Complementarily differential distribution of DNPI and BNPI immune reactivity in pain-relevant brain regions, such as the cingular cortex (Cg) and tectum and dorsal periaqueductal grey. DNPI dominance in the tectum and dorsal grey. Consecutive sections of a rat brain through the upper mesencephalon.

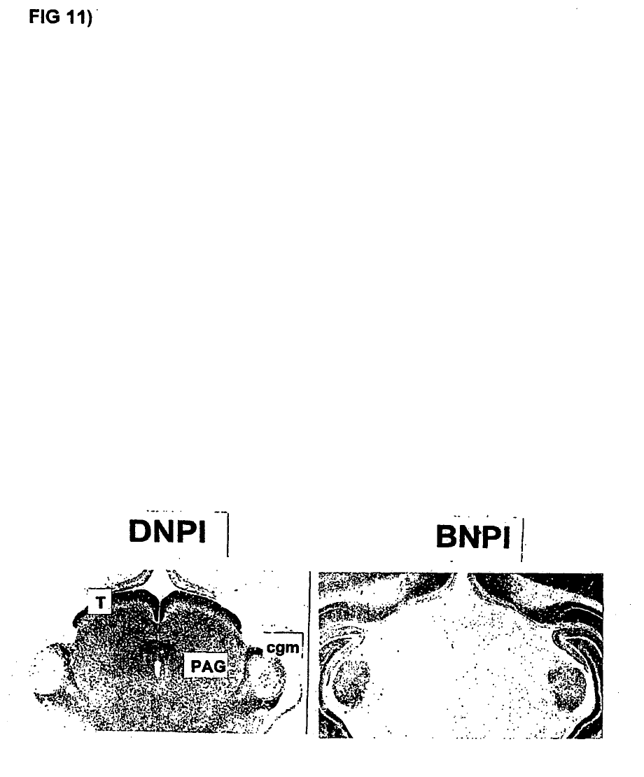

### Example 3g on FIG. 11)

Complementarily differential distribution of DNPI and BNPI immune reactivity in pain-relevant brain regions, such as the tectum (T) and periaqueductal grey (PAG). DNPI dominance in the tectum and dorsal grey. Differential distribution of DNPI and BNPI in the corpus geniculatum mediale (cgm) of the auditory path is to be noted. Consecutive sections of a rat brain through the upper mesencephalon; colliculus superior plane.

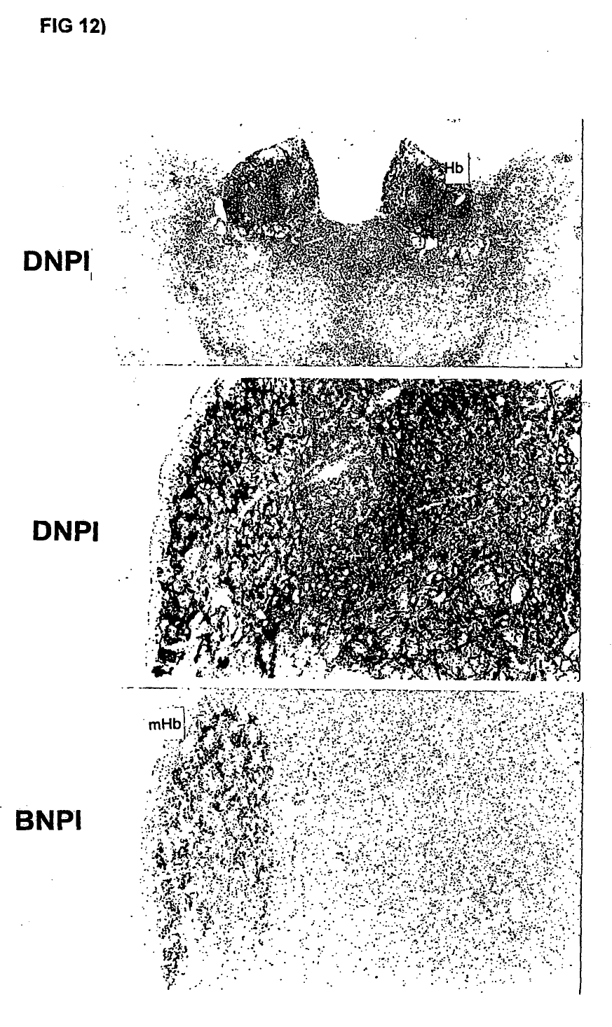

### Example 3h on FIG. 12)

Abundance of DNPI over BNPI in the habenulae (Hb). DNPI is present in the entire habenular complex (low magnification, upper figure; high magnification, middle fig.). BNPI is only in the medial habenular core (mHb lower fig., consecutive section to the middle figure).

Analysis of Example 3 Generally

The differential distribution of BNPI and DNPI in synapses of the primary afferent, spinal trigeminal and supraspinal nociceptive system is strong evidence of a selective influencability of nociceptive functions by selective modulation of the DNPI- or BNPI-mediated glutamate transport. The distribution of BNPI in the deep dorsal horn is an indication of a preferential role of BNPI in glutamate-driven neuropathic pain.

The preferential distribution of DNPI in lamina 1 and the substantia gelatinosa of the spinal and trigeminal nociceptive system suggests a primary and preferential role of DNPI in inflammation pain. Since DNPI synapses also lie in the deeper dorsal horn, DNPI is also a candidate in the case of neuropathic pain.

BNPI is a preferential candidate for allodynia and mechanical hyperalgesia with inflammation pain. Glutamate-mediated Aβ input converging on spinal nociceptive projection neurons could be a substantial mechanical for chronic deep musculoskeletal pain, a main problem of chronic pain.

The presence in visceral sacral afferences points to an indication in visceral pain.

Trigeminal afference: migraine, cluster headache, trigeminus neuralgia.

### Example 4

### Procedure for the Screening Method with Measurement of the Binding Via the Displacement of a Radioactively Labeled Ligand

A nucleic acid section which codes for BNPI is cloned in an expression vector which allows a constitutive expression (e.g. CMV promoter) or an inducible expression in eukaryotic cells. The DNA is introduced with suitable transfection processes, e.g. with Lipofectamin (Roche Diagnostics), into eukaryotic cells (e.g. CHO cells, HEK293 cells or NIH-3T3 cells). The cells are cultured in the presence of a selection reagent (e.g. zeocin, hygromycin or neomycin) such that only the cells which have taken up the DNA construct and, during longer-lasting selection, also incorporated it into the genome survive.

Starting from these cells, membrane fractions which contain BNPI in a large amount and can be used for a binding assay are obtained. This assay consists of 1.) the membranes containing BNPI, 2.) a radioactively labeled ligand, 3.) a binding buffer (e.g. 50 mM HEPES pH 7.4, 1 mM EDTA) and the ligand to be investigated for binding. After incubation of the abovementioned reaction mixtures (for e.g. 30-60 min) at a suitable temperature (usually room temperature), the non-bound radioactive ligand molecules are filtered off. The remaining amount of bound ligand is measured, after addition of a scintillation cocktail, in a β-counter (e.g. Trilux, Wallac). If the test substance shows binding to the BMPI, this is detected as a reduced radioactive incorporation. This method is suitably miniaturized such that it can be carried out in (96-, 384- or 1,536-well) microtiter plates in order to carry out this method by means of a robot in the so-called high throughput screening (HTS) method.

### Example 5

### Procedure for the Screening Method According to the Invention with BNPI and Measurement of the Functional Parameters Modified by Binding of the Substance

A nucleic acid section which codes for BNPI is cloned in an expression vector which allows an inducible expression in prokaryotes, such as e.g. E. coli. The nucleic acid section is modified here such that it is expressed as a fusion protein with an additional N- or C-terminal amino acid sequence. This sequence should allow, with a non-modified function of the BNPI, a purification via a specific method, e.g. glutathione S-transferase fragment, which allows isolation from the protein mixture via binding to glutathione. After transfection of the bacteria, induction of the gene (e.g. with IPTG in the case of the lac promoter) and breaking down of the bacteria, the fusion proteins are purified and employed in an in vitro kinase experiment. In this, 5 μg protein are at 30° C. for 30 minutes in 50 μl kinase buffer (20 mM PIPES, pH 7.0, 5 mM MnCl₂, 7 mM β-mercaptoethanol, 0.4 mM spermine, 10 mM rATP) supplemented with 10 μCi [γ³²P] ATP. Purified histone H1 protein (Sigma) or bacterially expressed GST-NFATc1 fusion protein are added as substrates. After the incubation time, the non-incorporated [γ-³²] ATP is filtered off and the amount of ³²phosphate incorporated is determined by β-scintillation (Trilux, Wallac). In an experiment for discovering new BNPI inhibitors, the test substances are co-incubated in this batch and a decrease in the ³²P incorporation is used as an indicator for an inhibitor. This method is suitably miniaturized such that it can be carried out in (96-, 384- or 1,536-well) microtiter plates in order to carry out this method by means of a robot in the so-called high troughput screening (HTS) method.

### Example 6

### Procedure for the Screening Method According to the Invention with DNPI and Measurement of the Functional Parameters Modified by Binding of the Substance

The method is carried out as described in example 5, with the exception that instead of a nucleic acid section which codes for BNPI, a nucleic acid section which codes for DNPI was employed.

### Example 7

### Example of a Pharmaceutical Formulation for Pain Treatment Comprising a Compound According to the Invention—Tablet Formulation

Tablets can be prepared by direct pressing of mixtures of the compound according to the invention with corresponding auxiliary substances or by pressing granules containing the compound (with optionally further auxiliary substances). The granules can be prepared here either by moist granulation with e.g. aqueous granulating liquids and subsequent drying of these granules or by dry granulation, e.g. via compacting

Direct pressing

| | | |
|---|---|---|
| e.g. per tablet: | 25 mg | compound according to the invention |
| | 271 mg | LudipressTM (granules for direct tablet |
| | | making from lactose monohydrate, |
| | | povidone K30 and crospovidone) |
| | 4 mg | magnesium stearate |
| | 300 mg | total |

Prepare a homogeneous mixture of the active compound with the auxiliary substances and press this on a tablet press to give tablets with a Ø of 10 mm.

Dry granulation

| | | |
|---|---|---|
| e.g. per tablet: | 25 mg | compound according to the invention |
| | 166 mg | microcrystalline cellulose |
| | 80 mg | hydroxypropylcellulose with a low degree |
| | | of substitution (I-HPC LH 11 ™) |
| | 5 mg | highly disperse silicon dioxide |
| | 4 mg | magnesium stearate |
| | 280 mg | total |

Prepare a homogeneous mixture of the compound with the microcrystalline cellulose and the I-HPC and copact this. After sieving of the compressed bodies, the granules formed are mixed with magnesium stearate and silicon dioxide and pressed on a tablet press to give tablets with a Ø of 9 mm.

Moist granulation

| | | |
|---|---|---|
| e.g. per tablet: | 25 mg | compound according to the invention |
| | 205 mg | microcrystalline cellulose |
| | 6 mg | povidone K30 |
| | 10 mg | crospovidone |
| | 4 mg | magnesium stearate |
| | 250 mg | total |

Prepare a homogeneous mixture of the compound with the microcrystalline cellulose and the crospovidone and granulate this with an aqueous solution of the povidone in a granulator. The moist granules are then after-granulated and, after drying, dried in a drying cabinet (50° C.) for 10 h. The dry granules are sieved together with the magnesium stearate, finally mixed and pressed on a tablet press to give tablets with a Ø of 8 mm.

### Example 8

### Example of a Pharmaceutical Formulation for Pain Treatment Comprising a Compound According to the Invention—Parenteral Solution

1 g of a compound according to the invention is dissolved in 1 l water for injection purposes at room temperature and the solution is then adjusted to isotonic conditions by addition of NaCl (sodium chloride).

The foregoing description and examples have been set forth merely to illustrate certain embodiments of the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

Literature:

The following documents are incorporated herein by reference in their entirety.

Literature

Aihara Y, Mashima H. Onda H. Hisano Setsuji, Kasuya H., Hori T. Yamada S., Tomura H. Yamada Y., Inoue I., Kojima I. and Takeda J. (2000), J. Neurochem. 74:2622-2625

Akopian A N, Sivilotti L & Wood J N (1995) Nature 379:257-262.

Ausubel F M, Brent R, Kingdton R E, Moore D D, Seidman J G, Smith J A & Struhl K eds. (1990) Current protocols in molecular biology. John Wiley & Sons, Inc. New York, N.Y.

Baba H, Doubell T P, Woolf C J 1999: Peripheral inflammation facilitates Aβ fiber-mediated synaptic input to the substantia gelatinosa of the adult rat spinal cord. J Neurosci 19:859-867.

Bauer D, Müller H, Reich J, Riedel H, Ahrenkiel V, Warthoe P & Strauss M (1993): Identification of differentially expressed mRNA species by an improved display technique (DDRT-PCR) Nucl Acids Res 21:4272-4280.

Bonini A, Anderson S M, Steiner D F (1997) Molecular cloning and tissue expression of a novel orphan G protein-coupled receptor from rat lung. Biochem Biophys Res Comm 234:190-193.

Chih-Cheng et al., (1995): A P2X prinoceptor expressed by a subset of sensory neurons. Nature 377:428-432.

Corderre T J, Katz J, Vaccarino A L, Melzack R (1993): Contribution of central plasticity to pathological pain: review of clinical and experimental evidence. Pain 52:259-285.

Dickenson (1995) Novel pharmacological targets in the treatment of pain. Pain Rev., 2, 1-12.

Dubuisson et al., 1997 Pain, 4:161-174.

Feng Y & Gregor P (1997) Cloning of a novel member of the G protein-coupled receptor family related to peptide receptors. Biochem Biophys Res Comm 231:651-654.

Furukawa T, Yang Y, Nakamoto B, Stamatoyannopoulos G, Papayannopoulou T (1996): Identification of new genes expressed in a human erythroleukemia cell line. Bloods Cell Mol & Dis 22:11-22.

Gunasekar P G, Kanthasamy, A G, Borowitz J L, Isom G E 1995: NMDA receptor activation produces concurrent generation of nitric oxide and reactive oxygen species: implication for cell death. J Neurochem 65:2016-2021.

Hawes B E, Fried S, Yao X, Weig B, Graziano M P 1998: Nociceptin (ORL1) and μ-opioid receptors mediate mitogen-activated protein kinase activation in CHO cells through a Gi-coupled signaling pathway: evidence for distinct mechanisms of agonist-mediated desensitization. J Neurochem 71:1024-1033.

Hubank M & Schatz D G (1994): Identifying differences in mRNA expression by representational difference analysis of cDNA. Nucl Acids Res 22:5640-5648.

Kluβmann S et al., 1996: Nature Biotechnology 14:1112-1115.

Li L-Y & Chang K-J 1996: The stimulatory effect of opioids on mitogen-activated protein kinase in Chinese hamster ovary cells transfected to express β-opioid receptors. Mol Pharm 50:599-602.

Lian P & Pardee A B 1992: Differential display of eukaryotic messenger RNA by means of the polymerase chain reaction. Science 257:967-971.

Methner A, Hermey G, Schinke B, Hermanns-Borgmeyer I (1997): A novel G protein-coupled receptor with homology to neuropeptide and chemoattractant receptors expressed during bone development. Biochem Biophys Res Comm 233:336-342.

Mohit A A, Martin J H & Miller C A 1995: p493F12 kinase: a novel MAP kinase expressed in a subset of neurons in the human nervous system. Neuron 14:67-78.

Poirier G M-C, Pyati J, Wan J S, Erlander M G 1997: Screening differentially expressed cDNA clones obtained by differential display using amplified RNA. Nucleic Acids Research 25:913-914.

Sambrook J, Fritsch E F & Maniatis T 1989: Molecular Cloning: A Laboratory Manual. Second Edition. Cold Spring Harbor Laboratory Press, Cold Spring Harbor.

Sompayrac L, Jane S, Burn T C, Tenen D G & Danna K J 1995: Overcoming limitations of the mRNA differential display technique. Nucleic Acids Research 23:4738-4739.

Tal M 1996: A novel antioxidant alleviates heat hyperalgesia in rats with an experimental painful neuropathy. Neurreport 7:1382-1384.

Tölle T R (1997): Chronischer Schmerz. In: Klinische Neurobiologie [Chronic Pain. In: Clinical Neurobiology], Herdergen T. Tölle T R, Bähr M (eds.): p. 307-336; Spektrum Verlag, Heidelberg.

U.S. Pat. No. 5,262,311 issued Nov. 16, 1993.

Velculescu V E, Zhang L, Vogelstein B, Kinzler K W (1995): Serial analysis of gene expression. Science 270:484-487.

Wan J S, Sharp J S et al. (1996): Cloning differentially expressed mRNAs. Nature Biotech 14:1685-1691.

Watson J B & Margulies J E (1993) Differential cDNA screening strategies to identify novel stage-specific proteins in the developing mammalian brain. Dev Neurosci 15:77-86.

Wilks A F (1989) Two putative protein-tyrosine kinases identified by application of the polymerase chain reaction. Proc Natl Acad Sci USA 86:1603-1607.

WO96/34288

Woolf C J, Shortland P, Coggeshall R E 1992: Peripheral nerve injury triggers central sprouting of myelinated afferents. Nature 355:75-78.

Zimmermann, M & Herdegen, T (1996): Plasticity of the nervous system at the systemic, cellular and molecular levels: a mechanism of chronic pain and hyperalgesia. Progr Brain Res 110:233-259.

What is claimed is:

1. A method for detecting a pain-regulating substance comprising the steps of:
(a) incubating a test substance with a biomolecule selected from group I, wherein group I consists of the protein BNPI or DNPI or a protein comprising SEQ ID NO: 2, 4, 6, 8, 10, 12 or 14 or a protein which is at least 90% homologous thereto or a protein encoded by a polynucleotide comprising SEQ ID NO: 1, 3, 5, 7, 9, 11 or 13 or a polynucleotide which is at least 90% homologous thereto, or a protein encoded by a nucleic acid which binds under stringent conditions to a polynucleotide comprising SEQ ID NO: 1, 3, 5, 7, 9, 11 or 13 or antisense polynucleotides thereof, or a part protein of one of the abovementioned proteins which is at least 10 amino acids long or a cell or a preparation from a cell which has synthesized at least one of the abovementioned proteins or part proteins and
(b) measuring the binding of the test substance to the protein or part protein synthesized by the cell or measuring at least one functional parameter modified by the binding of the test substance to the protein or part protein.

2. A method according to claim 1, wherein the cell is manipulated by genetic engineering before step (a).

3. A method according to claim 2, wherein the manipulation by genetic engineering allows the measurement of at least one functional parameter modified by the binding of the test substance.

4. A method according to claim 3, wherein the manipulation by genetic engineering causes expression of a form of a G protein which is not expressed endogenously in the cell or introduction of a reporter gene.

5. A method according to claim 2, wherein the cell is manipulated by genetic engineering so that the cell contains at least one polynucleotide selected from the group consisting of SEQ ID NOS: 1, 3, 5, 7, 9, 11 and 13, or a polynucleotide which is at least 90% homologous thereto.

6. A method according to claim 5, wherein the polynucleotide is contained in a recombinant DNA construct.

7. A method according to claim 2, wherein after the manipulation by genetic engineering according to claim 2 and before step (a), the cell is cultured under conditions which allow expression.

8. A method according to claim 7, wherein the cell is cultured under selection pressure.

9. A method according to claim 1, wherein the cell is an amphibia cell, bacteria cell, yeast cell, insect cell or an immortalized or native mammalian cell.

10. A method according to claim 1, wherein the measurement of the binding is carried out via the displacement of a known labeled ligand of the part protein or protein or via the activity bound thereto from a labeled test substance.

11. A method according claim 1, wherein the measurement of at least one functional parameter modified by the test substance is carried out via measurement of the regulation, inhibition or activation of receptors, ion channels or enzymes.

12. A method according claim 1, wherein the measurement of at least one functional parameter modified by the test substance is carried out via measurement of the modification of the gene expression, the ionic medium, the pH, the membrane potential, the enzyme activity or the concentration of the second messenger.

13. A method according to claim 1, in which a first method according to claim 1 is coupled with a second method according to claim 1 such that the measurement values and results of the first method in respect of the substance to be measured are compared with the measurement values and results of the second method in respect of the substance to be measured, wherein one of the two methods is the main method, wherein, in step (a) of said main method, the substance to be tested is incubated
either
with a biomolecule selected from group II, wherein group II consists of the the protein BNPI or a protein comprising SEQ ID NO: 2, 4, 6 or 8 or a protein which is at least 90% homologous thereto or a protein encoded by a polynucleotide comprising SEQ ID NO: 1, 3, 5 or 7 or a polynucleotide which is at least 90% homologous thereto, or a protein encoded by a nucleic acid which binds under stringent conditions to a polynucleotide comprising SEQ ID NO: 1, 3, 5 or 7 or antisense polynucleotides thereof, or a part protein of one of the abovementioned proteins which is at least 10 amino acids long or a cell or a preparation from a cell which has synthesized at least one of the abovementioned proteins or part proteins,
or
with a biomolecule selected from group III, wherein group III consists of the protein DNPI or a protein comprising SEQ ID NO: 10, 12 or 14, or a protein which at least 90% homologous thereto or a protein encoded by a polynucleotide comprising SEQ ID NO: 9, 11 or 13 or a polynucleotide which is at least 90% homologous thereto, or a protein encoded by a nucleic acid which binds under stringent conditions to a polynucleotide comprising SEQ ID NO: 9, 11 or 13 or antisense polynucleotides thereof, or a part protein of one of the abovementioned proteins which is at least 10 amino acids long or a cell or a preparation from a cell which has synthesized at least one of the abovementioned proteins or part proteins,
and
wherein, in a secondary method, in step (a) the substance to be tested is incubated with a biomolecule from group I or with a biomolecule selected from group II or group III, wherein the biomolecule selected from group II or group III is selected from a group which differs from group the biomolecule with which the substance in the main method is incubated.

14. A method according to claim 1, wherein the pain regulated by the substance to be detected is selected from the group consisting of:
chronic pain, neuropathic pain, mechanical hyperalgesia, diabetic neuropathy; visceral pain, cerebral pain, peripheral pain, inflammation-related pain, migraine, cluster headache or pain with trigeminus neuralgia.

15. The method of claim 14, wherein said pain is either musculoskeletal pain, allodynic pain or peripheral inflammation pain.

16. A compound identifiable as a pain-regulating substance by a method according to claim 1.

17. A compound according to claim 16, wherein said compound is a low molecular weight compound.

18. A method of alleviating pain comprising administering an effective pain alleviating amount of:
a. a polynucleotide which codes for BNPI or DNPI or a polynucleotide which is at least 90% homologous to one of the nucleotide sequences comprising SEQ ID NO: 1, 3, 5, 7, 9, 11 or 13,
b. a polynucleotide with a nucleotide sequence which is capable of binding specifically to one of the polynucleotides listed under point a),
c. a vector containing a polynucleotide according to one of points a) or b),
d. BNPI or DNPI or a protein comprising SEQ ID NO: 2, 4, 6, 8, 10, 12 or 14 or a protein which is at least 90% homologous thereto or a protein encoded by a polynucleotide comprising SEQ ID NO: 1, 3, 5, 7, 9, 11 or 13) or a polynucleotide which is at least 90% homologous thereto, or a protein encoded by a nucleic acid which binds under stringent conditions to a polynucleotide comprising SEQ ID NO: 1, 3, 5, 7, 9, 11 or 13 or antisense polynucleotides thereof or a part protein of one of the abovementioned proteins which is at least 10 amino acids long,
e. an antibody, against one of the proteins or part proteins according to point d),
f. a cell, containing a polynucleotide according to one of points a) or b), a vector according to point c), a protein or part protein according to point d) or an antibody according to point e)
g. a compound according to claim 16 or
h. an active compound, preferably a low molecular weight active compound, which binds to a protein or part protein according to point a), and
at least one carrier or auxiliary substance.

19. A method according to claim 18, wherein the pain is selected from the group consisting of:
chronic pain, neuropathic pain, mechanical hyperalgesia, diabetic neuropathy; visceral pain, cerebral pain, peripheral pain, inflammation-related pain, migraine, cluster headache or pain with trigeminus neuralgia.

20. The method of claim 19, wherein said pain is either musculoskeletal pain, allodynic pain or peripheral inflammation pain.

21. A method of providing gene therapy to a mammal, said method comprising administering to said mammal a therapeutic amount of:
a. a polynucleotide which codes for BNPI or DNPI or a polynucleotide which is at least 90% homologous to a nucleotide sequence comprising SEQ ID NO: 1, 3, 5 or 7,
b. a polynucleotide which is capable of binding specifically to one of the polynucleotides listed under point a),
c. a vector comprising a polynucleotide according to one of points a) or b), or
f. a cell containing a polynucleotide according to one of points a) or b) or a vector according to point c).

22. The method of claim 21, wherein said gene therapy is in vivo gene therapy.

23. The method of claim 21, wherein said gene therapy in vitro gene therapy.

24. The method of claim 21, wherein said gene therapy is effective in alleviating pain.

25. A method according to claim 21, wherein the pain is selected from the group consisting of:
chronic pain, neuropathic pain, mechanical hyperalgesia, diabetic neuropathy; visceral pain, cerebral pain, peripheral pain, inflammation-related pain, migraine, cluster headache or pain with trigeminus neuralgia.

26. The method of claim 25, wherein said pain is either musculoskeletal pain, allodynic pain or peripheral inflammation pain.

27. A method of diagnosing a pain state comprising administering an effective amount of a diagnostic agent comprising:
a. a polynucleotide which codes for BNPI or DNPI or a polynucleotide which is at least 90% homologous to a nucleotide sequence comprising SEQ ID NO: 1, 3, 5, 7, 9, 11 or 13,
b. a polynucleotide capable of binding specifically to one of the polynucleotides listed under point a),
c. a vector comprising a polynucleotide according to one of points a) or b),
d. BNPI or DNPI or a protein comprising SEQ ID NO: 2, 4, 6, 8, 10, 12 or 14 or a protein which at least 90% homologous thereto or a protein encoded by a polynucleotide comprising SEQ ID NO: 1, 3, 5, 7, 9, 11 or 13 or a polynucleotide which is at least 90% homologous thereto, or a protein encoded by a nucleic acid which binds under stringent conditions to a polynucleotide comprising SEQ ID NO: 1, 3, 5, 7, 9, 11 or 13 or antisense polynucleotides thereof or a part protein of one of the abovementioned proteins which is at least 10 amino acids long,
e. an antibody against one of the proteins or part proteins according to point d),
f. a cell containing a polynucleotide according to one of points a) or b), a vector according to point c), a protein or part protein according to point d) or an antibody according to point e)
g. a compound according to claim 16 or
h. an active compound, preferably a low molecular weight active compound, which binds to a protein or part protein according to point a), and
detecting an indicator affected by the diagnostic agent.

28. A method according to claim 27, wherein the pain state is selected from the group consisting of:
chronic pain, neuropathic pain, mechanical hyperalgesia, diabetic neuropathy; visceral pain, cerebral pain, peripheral pain, inflammation-related pain, migraine, cluster headache or pain with trigeminus neuralgia.

29. The method of claim 28, wherein said pain state is either musculoskeletal pain, allodynic pain or peripheral inflammation pain.

30. A method of detecting a pain-regulating substance comprising administering an effective amount of a diagnostic agent comprising:
a. a polynucleotide, which codes for BNPI or DNPI or a polynucleotide, which is at least 90% homologous with a nucleotide sequence comprising SEQ ID NO: 1, 3, 5, 7, 9, 11 or 13,
b. a polynucleotide which is capable of binding specifically to one of the polynucleotides listed under point a),
c. a vector containing a polynucleotide according to one of points a) or b),
d. BNPI or DNPI or a protein comprising SEQ ID NO: 2, 4, 6, 8, 10, 12 or 14 or a protein which at least 90% homologous thereto or a protein encoded by a polynucleotide comprising SEQ ID NO: 1, 3, 5, 7, 9, 11 or 13 or a polynucleotide which at least 90% homologous thereto, or a protein which encoded by a nucleic acid which binds under stringent conditions to a polynucleotide comprising SEQ ID NO: 1, 3, 5, 7, 9, 11 or 13 or antisense polynucleotides thereof or a part protein of one of the abovementioned proteins which is at least 10 amino acids long,
e. an antibody against one of the proteins or part proteins according to point d),
f. a cell containing a polynucleotide according to one of points a) or b), a vector according to point c), a protein or part protein according to point d) or an antibody according to point e), and
detecting an indicator affected by the diagnostic agent.

31. A method according to claim 30, wherein the pain regulated by said pain-regulating substance is selected from the group consisting of:
chronic pain, neuropathic pain, mechanical hyperalgesia, diabetic neuropathy; visceral pain, cerebral pain, peripheral pain, inflammation-related pain, migraine, cluster headache or pain with trigeminus neuralgia.

32. The method of claim 31, wherein said pain is either musculoskeletal pain, allodynic pain or peripheral inflammation pain.


SEQUENCE LISTING (14 sequences)

SEQ ID NO: 1 - DNA, 2366 bp, Homo sapiens
ccggcggcaggagccgccaccatggagttccgccaggaggagtttcggaagctagcgggtcgtgctctcgggaagctgcaccgccttctggagaagcggcaggaaggcgcggagacgctggagctgagtgcggatgggcgcccggtgaccacgcagacccgggacccgccggtggtggactgcacctgcttcggcctccctcgccgctacattatcgccatcatgagtggtctgggcttctgcatcagctttggcatccgctgcaacctgggcgtggccatcgtctccatggtcaataacagcacgacccaccgcgggggccacgtggtggtgcagaaagcccagttcagctgggatccagagactgtcggcctcatacacggctcctttttctggggctacattgtcactcagattccaggaggatttatctgtcaaaaatttgcagccaacagagttttcggctttgctattgtggcaacatccactctaaacatgctgatcccctcagctgcccgcgtccactatggctgtgtcatcttcgtgaggatcctgcaggggttggtagagggggtcacataccccgcctgccatgggatctggagcaaatgggccccacccttagaacggagtcgcctggcgacgacagccttttgtggttcctatgctggggcggtggtcgcgatgcccctcgccggggtccttgtgcagtactcaggatggagctctgttttctacgtctacggcagcttcgggatcttctggtacctgttctggctgctcgtctcctacgagtcccccgcgctgcaccccagcatctcggaggaggagcgcaagtacatcgaggacgccatcggagagagcgcgaaactcatgaaccccctcacgaagtttagcactccctggcggcgcttcttcacgtctatgccagtctatgccatcatcgtggccaacttctgccgcagctggacgttctacctgctgctcatctcccagcccgcctacttcgaagaagtgttcggcttcgagatcagcaaggtaggcctggtgtccgcgctgccccacctggtcatgaccatcatcgtgcccatcggcggccagatcgcggacttcctgcggagccgccgcatcatgtccaccaccaacgtgcgcaagttgatgaactgcggaggcttcggcatggaagccacgctgctgttggtggtcggctactcgcactccaagggcgtggccatctccttcctggtcctagccgtgggcttcagcggcttcgccatctctgggttcaacgtgaaccacctggacatagccccgcgctacgccagcatcctcatgggcatctccaacggcgtgggcacactgtcgggcatggtgtgccccatcatcgtgggggccatgactaagcacaagactcgggaggagtggcagtacgtgttcctaattgcctccctggtgcactatggaggtgtcatcttctacggggtctttgcttctggagagaagcagccgtgggcagagcctgaggagatgagcgaggagaagtgtggcttcgttggccatgaccagctggctggcagtgacgacagcgaaatggaggatgaggctgagcccccgggggcaccccctgcacccccgccctcctatggggccacacacagcacatttcagccccccaggcccccaccccctgtccgggactactgaccatgtgcctcccactgaatggcagtttccaggacctccattccactcatctctggcctgagtgacagtgtcaaggaaccctgctcctctctgtcctgcctcaggcctaagaagcactctcccttgttcccagtgctgtcaaatcctctttccttcccaattgcctctcaggggtagtgaagctgcagactgacagtttcaaggatacccaaattcccctaaaggttccctctccacccgttctgcctcagtggtttcaaatctctcctttcagggctttatttgaatggacagttcgacctcttactctctcttgtggttttgaggcacccacaccccccgctttcctttatctccagggactctcaggctaacctttgagatcactcagctcccatctcctttcagaaaaattcaaggtcctcctctagaagtttcaaatctctcccaactctgttctgcatcttccagattggtttaaccaattactcgtccccgccattccagggattgattctcaccagcgtttctgatggaaaatggcggtttcaagtccccgattccgtgcccacttcacatctcccctaccagcagattctgcgaaagcaccaaatttctcaagaccctcttctccctagcttagcataatgtctggggaaaca

SEQ ID NO: 2 - protein, 560 aa, Homo sapiens
MEFRQEEFRKLAGRALGKLHRLLEKRQEGAETLELSADGRPVTTQTRDPPVVDCTCFGLPRRYIIAIMSGLGFCISFGIRCNLGVAIVSMVNNSTTHRGGHVVVQKAQFSWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPLTKFSTPWRRFFTSMPVYAIIVANFCRSWTFYLLLISQPAYFEEVFGFEISKVGLVSALPHLVMTIIVPIGGQIADFLRSRRIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDDSEMEDEAEPPGAPPAPPPSYGATHSTFQPPRPPPPVRDY

SEQ ID NO: 3 - DNA, 2716 bp, Homo sapiens
cgataagcttgatatcgaattccggactcttgctcgggcgccttaacccggcgttcggttcatcccgcagcgccagttctgcttaccaaaagtggcccactaggcactcgcattccacgcccggctccacgccagcgagccgggcttcttacccatttaaagtttgagaataggttgagatcgtttcggccccaagacctctaatcattcgctttaccggataaaactgcgtggcgggggtgcgtcgggtctgcgagagcgccagctatcctgagggaaacttcggagggaaccagctactagatggttcgattagtctttcgcccctatacccaggtcggacgaccgatttgcacgtcaggaccgctacggacctccaccagagtttcctctggcttcgccctgcccaggcgatcggcgggggggacccgcggggtgaccggcggcaggagccgccaccatggagttccgccaggaggagtttcggaagctagcgggtcgtgctctcgggaagctgcaccgccttctggagaagcggcaggaaggcgcggagacgctggagctgagtgcggatgggcgcccggtgaccacgcagacccgggacccgccggtggtggactgcacctgcttcggcctccctcgccgctacattatcgccatcatgagtggtctgggcttctgcatcagctttggcatccgctgcaacctgggcgtggccatcgtctccatggtcaataacagcacgacccaccgcgggggccacgtggtggtgcagaaagcccagttcagctgggatccagagactgtcggcctcatacacggctcctttttctggggctacattgtcactcagattccaggaggatttatctgtcaaaaatttgcagccaacagagttttcggctttgctattgtggcaacatccactctaaacatgctgatcccctcagctgcccgcgtccactatggctgtgtcatcttcgtgaggatcctgcaggggttggtagagggggtcacataccccgcctgccatgggatctggagcaaatgggccccacccttagaacggagtcgcctggcgacgacagccttttgtggttcctatgctggggcggtggtcgcgatgcccctcgccggggtccttgtgcagtactcaggatggagctctgttttctacgtctacggcagcttcgggatcttctggtacctgttctggctgctcgtctcctacgagtcccccgcgctgcaccccagcatctcggaggaggagcgcaagtacatcgaggacgccatcggagagagcgcgaaactcatgaaccccctcacgaagtttagcactccctggcggcgcttcttcacgtctatgccagtctatgccatcatcgtggccaacttctgccgcagctggacgttctacctgctgctcatctcccagcccgactacttcgaagaagtgttcggcttcgagatcagcaaggtaggcctggtgtccgcgctgccccacctggtcatgaccatcatcgtgcccatcggcggccagatcgcggacttcctgcggagccgccgcatcatgtccaccaccaacgtgcgcaagttgatgaactgcggaggcttcggcatggaagccacgctgctgttggtggtcggctactcgcactccaagggcgtggccatctccttcctggtcctagccgtgggcttcagcggcttcgccatctctgggttcaacgtgaaccacctggacatagccccgcgctacgccagcatcctcatgggcatctccaacggcgtgggcacactgtcgggcatggtgtgccccatcatcgtgggggccatgactaagcacaagactcgggaggagtggcagtacgtgttcctaattgcctccctggtgcactatggaggtgtcatcttctacggggtctttgcttctggagagaagcagccgtgggcagagcctgaggagatgagcgaggagaagtgtggcttcgttggccatgaccagctggctggcagtgacgacagcgaaatggaggatgaggctgagcccccgggggcaccccctgcacccccgccctcctatggggccacacacagcacatttcagccccccaggcccccaccccctgtccgggactactgaccatgtgcctcccactgaatggcagtttccaggacctccattccactcatctctggcctgagtgacagtgtcaaggaaccctgctcctctctgtcctgcctcaggcctaagaagcactctcccttgttcccagtgctgtcaaatcctctttccttcccaattgcctctcaggggtagtgaagctgcagactgacagtttcaaggatacccaaattcccctaaaggttccctctccacccgttctgcctcagtggtttcaaatctctcctttcagggctttatttgaatggacagttcgacctcttactctctcttgtggttttgaggcacccacaccccccgctttcctttatctccagggactctcaggctaacctttgagatcactcagctcccatctcctttcagaaaaattcaaggtcctcctctagaagtttcaaatctctcccaactctgttctgcatcttccagattggtttaaccaattactcgtccccgccattccagggattgattctcaccagcgtttctgatggaaaatggcgggaattcctgcagcccgggggatccact

SEQ ID NO: 4 - protein, 560 aa, Homo sapiens
MEFRQEEFRKLAGRALGKLHRLLEKRQEGAETLELSADGRPVTTQTRDPPVVDCTCFGLPRRYIIAIMSGLGFCISFGIRCNLGVAIVSMVNNSTTHRGGHVVVQKAQFSWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPLTKFSTPWRRFFTSMPVYAIIVANFCRSWTFYLLLISQPDYFEEVFGFEISKVGLVSALPHLVMTIIVPIGGQIADFLRSRRIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDDSEMEDEAEPPGAPPAPPPSYGATHSTFQPPRPPPPVRDY

SEQ ID NO: 5 - DNA, 2024 bp, Rattus norvegicus
gaattcggcacgagcggagctgcggggccgggccgggccggggcggaccccgggatcccggacgcggccgcccgggcccgcgggcggggggattggcaggggacccgcgtgggcacagccaccatggagttccggcaggaggagtttcggaagctggcggggcgcgccctggggaggctgcaccggttactggagaagcggcaggaaggcgcggagacattggagctgagcgccgacgggcgcccagtgaccacacacacgcgggacccgccggtggtggactgcacttgctttggcctccctcgccgctacatcatcgcgatcatgagcggtctgggtttctgcatcagctttggcatccgctgcaacctgggcgtggccatcgtatccatggtcaacaacagtacaacccaccgtgggggccacgtggtggtgcagaaagcccagttcaactgggatccagagactgtcggcctcatacatggctcctttttctgggggtacattgtcactcagattcctggaggatttatctgccaaaaattcgcagccaacagggtctttggctttgccattgtggctacctccaccctaaatatgttgatcccttcagcagcccgtgttcactatggctgtgtcatcttcgtgaggatccttcagggattggtggagggggtcacataccctgcttgccatggcatctggagcaaatgggcccctcccttagaacggagtcggctggcgacgacagccttttgcggttcctatgccggggcagtggttgccatgcctctggctggggtcctggtacagtattcaggatggagttctgtcttctatgtctatggcagcttcgggatcttttggtacctgttctggttgcttgtctcctacgagtcacctgcactacaccccagcatctccgaggaggagcgcaaatacattgaggatgccatcggagaaagcgccaagctcatgaaccctgttacgaagtttaacacaccctggaggcgcttctttacctccatgccggtctatgccatcattgtcgccaacttttgccgcagctggactttctacctgctcctcatctcccagcccgcctactttgaagaagtgttcggctttgagatcagcaaggtgggactggtgtcggcactgcctcaccttgtcatgactatcatcgtacccatcggaggccagatcgccgacttcctgcgcagtcgtcatataatgtccacgaccaatgtgcgaaagctgatgaactgcgggggtttcgggatggaagctacgctgctgctggtggtcggatactcacactccaagggcgtggccatctccttcctggtcctggctgtgggcttcagtggctttgctatctctgggtttaacgtgaaccacttggacatcgcccctcgatatgccagcatcttgatgggcatttccaatggcgtgggcacactgtctgggatggtgtgccccatcatcgtgggtgcaatgaccaagcacaagacgcgggaggagtggcagtacgtgttcctcatagcctccctggtgcactatggaggtgtcatcttctatggggtctttgcttcgggagagaaacagccgtgggcagagccggaggagatgagcgaggagaagtgtggctttgttggccacgaccagctggctggcagtgacgaaagtgaaatggaagacgaggttgagcccccgggggcaccccccgcacctccgccttcctacggggccacacacagcacagttcagcctccaaggcccccaccccctgtccgggactactgaccacgtgcctcccactggtgggcagtttccaggacctccactcgatacacctctagcctaaacggcagtgtcgaggaaccccactcctctcctgcctcaggcttaagatgcaagtcttcccttgtgcccagtgctgtccgaccagccctctctccttctcaactgcctcttgcaggggtgaagctgcacactagcagtttcaagctcgtgccgaattc

SEQ ID NO: 6 - protein, 560 aa, Rattus norvegicus
MEFRQEEFRKLAGRALGRLHRLLEKRQEGAETLELSADGRPVTTHTRDPPVVDCTCFGLPRRYIIAIMSGLGFCISFGIRCNLGVAIVSMVNNSTTHRGGHVVVQKAQFNWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPVTKFNTPWRRFFTSMPVYAIIVANFCRSWTFYLLLISQPAYFEEVFGFEISKVGLVSALPHLVMTIIVPIGGQIADFLRSRHIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDESEMEDEVEPPGAPPAPPPSYGATHSTVQPPRPPPPVRDY

SEQ ID NO: 7 - DNA, 2836 bp, Mus musculus
cggccgcccgggcccgcgggcggggggattggcaggggacccgcgtgggcacagccaccatggagttccggcaggaggagtttcggaagctggcggggcgcgccctggggaggctgcaccggttactggagaagcggcaggaaggcgcggagacactggagctgagtgccgacgggcggccagtgaccacgcacactcgggacccgcctgtggtggactgcacctgctttggcctccctcgtcgctacatcatcgccatcatgagcggtctgggtttctgtatcagctttggcatccgctgcaacctgggcgtggccatcgtgtccatggtcaacaacagcacaacccaccgtgggggccacgtggtggtgcagaaagcccagttcaactgggatccagagactgtcggcctcatacatggctcctttttctggggctacattgtcactcagattcctggaggatttatctgccaaaaattcgcagccaacagggtctttggctttgccattgtggctacctccaccctaaacatgttgatcccttcagcagcccgcgttcactatggctgtgtcatcttcgtgaggatccttcagggattggtggagggggtcacataccctgcttgccatggcatctggagcaaatgggcccctcccttagaacggagtcggctggcaacgacagccttttgcggttcctatgctggggcggtggttgccatgcccttggctggggtccttgtgcagtattcaggatggagttctgtcttctatgtctatggcagcttcgggatcttttggtacctgttctggttgcttgtctcctatgagtcaccggcactgcaccccagcatctctgaggaggagcgcaaatacattgaggatgccatcggggagagcgccaagctcatgaaccctgttacgaagtttaacacaccctggaggcgcttctttacgtccatgcccgtctatgccatcatcgttgcgaacttttgccgcagctggaccttctacctgctcctcatctctcagcccgcctactttgaagaagtgttcggctttgagatcagcaaggtggggctggtgtcggcgctgcctcaccttgtcatgaccatcatcgtacccattggaggccagatcgctgactttttgcgcagtcgtcacataatgtccactaccaacgtgcgaaagctcatgaactgcgggggtttcgggatggaagccacgctgctgctggtggtcggatactcgcactccaagggcgtggccatctccttcctggtcctggctgtgggcttcagtggctttgccatctctgggtttaacgtgaaccacttggacatcgcccctcgctatgccagcatcttgatgggcatttccaatggcgtgggcacactgtctgggatggtgtgccccatcatcgtgggtgcaatgaccaagcacaagacgcgggaggagtggcagtacgtgttcctcatagcctccctggtgcactacggcggtgtcatcttctatggggtctttgcttcgggagagaagcagccgtgggcagagccggaggagatgagcgaggagaagtgtggctttgttggccacgaccagctggctggcagtgacgaaagtgaaatggaggacgaggctgagcccccaggggcgccccccgcgccgcctccgtcctacggggccacacacagcacagtgcagcctccgaggcccccgccccctgtccgggactactgaccacgggcctcccactgtggggcagtttccaggacttccactccatacacctctagcctgagcggcagtgtcgaggaaccccactcctcccctgcctcaggcttaagatgcaagtcctcccttgttcccagtgctgtccgaccagccctctttccctctcaactgcctcctgcggggggtgaagctgcacactagcagtttcaaggatacccagactcccctgaaagtcgttctccgcttgtttctgcctgtgtgggctcaaatctcccctttgagggctttatttggagggacagttcaacctcttcctctcttgtggttttgaggtttcaccccttcccccaagaccccagggattctcaggctaccccgagattattcaggtggtcccctactcagaagacttcatggtcgtcctctattagtttcaaggctcgcctaaccaattctacatttttccaagctggtttaacctaaccaccaatgccgccgttcccaggactgattctcaccagcgtttctgagggaaaatggcggtttcaagtccccccaccccccttttcttccctcgtcccctcaccagcacactttgccgggccttgaccttagcttagtacaatcattgtccagggaaatggccaaaatggctctgctcaccccgtgctctttttctgactcagttttcaggtctcagtagtggctgcccaaagctattaattcagcggctcgaggccacctcttcctccccgtggtggtttcaggatccccctcgcccccccccccccaaatccttgcactttattctcctgggtggttccaggccgccctcggtttctcagtggccatttgttgtgtgtccctcaggggctaaatgattccaaatctggggtgcttcccctcatagacacccctctctcaacgtagaaatctgggtgggggtgaggtgtgtgagagaagttacagaatcccaggaaagggagcggggctgggaggagagggttgttcctggggcagggtcgtgtcttggtgtctgtctctgtgacgtaaatcctgccctgccccaccccactcccaataaacgctctggtgtacgg

SEQ ID NO: 8 - protein, 560 aa, Mus musculus
MEFRQEEFRKLAGRALGRLHRLLEKRQEGAETLELSADGRPVTTHTRDPPVVDCTCFGLPRRYIIAIMSGLGFCISFGIRCNLGVAIVSMVNNSTTHRGGHVVVQKAQFNWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPVTKFNTPWRRFFTSMPVYAIIVANFCRSWTFYLLLISQPAYFEEVFGFEISKVGLVSALPHLVMTIIVPIGGQIADFLRSRHIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDESEMEDEAEPPGAPPAPPPSYGATHSTVQPPRPPPPVRDY

SEQ ID NO: 9 - DNA, 3946 bp, Homo sapiens
cgtttaaaagccatcagatttgagagcaataagtcttcaaaaccgggaatttacattgtttttcagctgaccgacttccaggaaaaggactcaaccgcatctacccaaataccgtggcactgcttgcgctctttgccaccggatactccccttccaatgagactttctgattgtgtctaccaactctcctattaggaaacccgtgggttgcatgcagctattctgttgtattctcattctcactctccctcccttctctcactctcactcttgctggaggcgagccactaccattctgctgagaaggaaaagcccgcaactactttaagagattaagacaatatgcgcaatcctcgcctttcctagcaatcactatttaaatctggcaagaactgacaacagtctttgcaagaatggaatccgtaaaacaaaggattttggccccaggaaaagaggggctaaagaattttgctggaaaatcactcggccagatctacagggtgctggagaagaagcaagacaccggggagacaatcgagctgacggaggatgggaagcccctagaggtgcccgagaggaaggcgccgctgtgcgactgcacgtgcttcggcctgccccgccgctacattatcgccatcatgagcggcctgggcttctgcatctccttcggtatccgctgcaacctgggcgtggccattgtggacatggtcaacaacagcaccatccaccgcgggggcaaggtcatcaaggagaaagccaaattcaactgggacccggaaaccgtggggatgatccacggttccttcttttggggctacatcatcactcagattccgggaggctacatcgcgtctcggctggcagccaacagggttttcggagctgccatacttcttacctctaccctaaatatgctaattccatcagcagccagagtgcattatggatgtgtcatctttgtcagaatactgcagggacttgttgagggtgtgacctacccagcatgtcatgggatatggagcaaatgggccccacctctagagaggagtagactggcaaccacctccttttgtggttcctatgccggagctgtgattgcaatgcctttagctggcattcttgtgcagtacactggctggtcttcagtgttttatgtctacggaagctttggaatggtctggtacatgttttggcttttggtgtcttatgaaagtcctgcaaagcatcctactattacagatgaagaacgtaggtacatagaagaaagcattggagagagtgcaaatcttttaggtgcaatggaaaaattcaagactccatggaggaagttttttacatccatgccagtctatgcaataattgttgcaaacttctgcagaagctggactttttatttattgcttattagtcagccagcatattttgaggaagtctttggatttgaaattagcaaggttggtatgctatctgctgtgccacacttagtaatgacaattattgtgcctattgggggacaaattgcagattttctaagaagcaagcagattctttcaactacgacagtgagaaagatcatgaattgtggtggttttggcatggaagccacactgctcctggtcgttggctattctcatactagaggggtagcaatctcattcttggtacttgcagtgggattcagtggatttgctatatctggtttcaatgttaaccacttggatatcgctccaagatatgccagtatcttaatgggcatttcgaatggtgttggcacattgtcaggaatggtttgtcctatcattgttggtgcaatgacaaagaataagtcacgtgaagagtggcagtatgtcttcctgatcgctgccctagtccactatggtggagttatattttatgcaatatttgcctcaggagagaaacaaccctgggcagacccggaggaaacaagtgaagaaaaatgtggatttattcatgaagatgaactcgatgaagaaacaggggacattactcaaaattatataaattatggtaccaccaagtcttatggtgccacaacacaggccaatggaggttggcctagtggttgggaaaagaaagaggaatttgtacaaggagaagtacaagactcacatagctataaggaccgagttgattattcataacaaaactaattactggatttatttttagtgtttgtgattaaattcattgtgattgcacaaaaattttaaaaacacgtgatgtaaacttgcaagcatatcaaccaggcaagtcttgctgtaaaaatgaaaacaaaacaaacccatgaggttaccatcaagtgcaatctgtaaaattgtgaagttccatcatttccattcaagtcatccattcttgcatttgtgacttaaaggttgactggtcaaaattgtagaaacaagtagttacccattggattcatatgagctaaaactcatcactatttactaaagcacaacatctcatcctacaaaagttaagaagccaaagctacttgatcatgcaaaatgcacttatatatttgttacactgtattgcaagatagcacacagaagttggctgcgtcaagtagaggcgacatttattaagtgaaaatcatggagttgggatatctctcaattaaagaaatacattgtgaactatcagctacaaagttgtactgaataactattagaattgcataatgtgagatattttgttagtcctcaaaaggaatatcttgcagtgttttctatgaaatgcttgggcacaaacacttatttctgtgaaagagaacatgtaagttgaggggtatgcttcatgttcttccatccatttacctaatagtatgaaacagttcacatttcaataaaatcaaacttttcatgtagcgtatcacataacttttttgcaaaaaatataaaaagaaataaacttcaatgtattttttattacaactttgtactggttgtaacttgcattagaaaaaaaaaagagatatataaaccacaaagaatctaataagaaatttattatggagatatagcccttaaaatgcaatattaagaacaaagaaatagaaaatggtttagatatctttcttccttcataattaaatactatatgaaacttgtgccacagagctatatgtaatatgaaaagattaacttcatagagatattgtaagtaggtaattttattatttaaagtcctattaagaaatatttgtcttaaatatataggacaatacattatattaaaatggtctctctctatatatatctgtatatcttatacatgtccatacacagaaacataataaacaatcttcacacgaaaccaaaaatagcatacacctaatgttgggttagggaattgcaatttctactttcatagagtcatagaattttaggtggggaagaggcattttgcttgtcatttcttaatataactcaacaagaattgcaacatttgtgtaccaagcaataagtgcaatgcataaaatttcctgtctgtatattaccttcattttgcttgtagtagctgtttgggtggttggaataattttatttttcttttaaaaaagctaacatcagacccctttataatgtcctaaaattatgataatacatttcccaattcaactcaaaatattattggtgtattttgtctattctggatatttgatctgtttaatgtactgtgctagtgactggaggccctgctactgcaaatataaaacctaaagtttgtttaaaaaaatgcaaatcattctttaccttaagaaaaaaaaaataccctttgctttgtgcctcaaagtgatgtaatgtgatcacagcttttgttgtgttgaatgaaaatatgtggactgtcattttgttgcagcaaaaaagtgttaataaaatgctctatttatccttttttaaaaaaaaaaaaaaaaaaaaaaaaa

SEQ ID NO: 10 - protein, 582 aa, Homo sapiens
MESVKQRILAPGKEGLKNFAGKSLGQIYRVLEKKQDTGETIELTEDGKPLEVPERKAPLCDCTCFGLPRRYIIAIMSGLGFCISFGIRCNLGVAIVDMVNNSTIHRGGKVIKEKAKFNWDPETVGMIHGSFFWGYIITQIPGGYIASRLAANRVFGAAILLTSTLNMLIPSAARVHYGCVIFVRILQGLVEGVTYPACHGIWSKWAPPLERSRLATTSFCGSYAGAVIAMPLAGILVQYTGWSSVFYVYGSFGMVWYMFWLLVSYESPAKHPTITDEERRYIEESIGESANLLGAMEKFKTPWRKFFTSMPVYAIIVANFCRSWTFYLLLISQPAYFEEVFGFEISKVGMLSAVPHLVMTIIVPIGGQIADFLRSKQILSTTTVRKIMNCGGFGMEATLLLVVGYSHTRGVAISFLVLAVGFSGFAISGFNVNHLDIAPRYASILMGISNGVGTLSGMVCPIIVGAMTKNKSREEWQYVFLIAALVHYGGVIFYAIFASGEKQPWADPEETSEEKCGFIHEDELDEETGDITQNYINYGTTKSYGATTQANGGWPSGWEKKEEFVQGEVQDSHSYKDRVDYS

SEQ ID NO: 11 - DNA, 3982 bp, Rattus norvegicus
agacagtaaggttcttttgcttttttcccttacacaaggattcgatgacgtttttggtcaatctgattaaaagacagcggatttggttgcgttaagacttcaaaaccgggaatttacgttgtttttcggtgaggtgacttccagaacggggactcatcagcacccgcccaaataccacggcactgcgcgcgccctcggccaccggatcctccccttccaatgagactttgtgactgtgtgtaccaattctcctattaggaaacccgtgggctgaatgcagctattccgttgtactctctttctcgctctccctcccctctccaactcacagccttgctgaaaagctcatctctgctgagaagaaaacgttctaccttaacctattaagactatgcgcagaactcgcctttcatagccatcacaatttaaatctggtaaggctggacacgagtctttacaagaatggagtcggtaaaacaaaggattttggccccggggaaagaggggataaagaattttgctggaaaatccctcggacagatctacagggtgctggagaagaagcaggataaccgagagaccatcgagctgacagaggacggcaagcccctggaggtgcctgagaagaaggctccgctatgcgactgtacgtgcttcggcctgccgcgccgctacatcatagccatcatgagcggcctcggcttctgcatctcctttggtatccgctgtaacctgggtgtggccattgtggacatggtcaacaacagcaccatccaccggggtggcaaagttatcaaggagaaagccaagtttaactgggaccccgagactgtggggatgattcacgggtcgttcttctggggctatatcatcacgcagattccgggcggatacatcgcatcgcgactggctgctaaccgggtctttggggctgccatactgcttacctctaccctcaatatgctgatcccatctgcagccagagtgcattatggatgcgtcatctttgttagaatattgcaaggacttgtggagggcgtcacctacccagcctgtcacgggatatggagcaagtgggcccctcctttggagaggagtaggttggctaccacctccttctgtggttcctatgctggagcagtcattgcaatgcccctagctggtatcctggtgcagtacactggatggtcttcagtattttacgtatatggaagctttggtatggtctggtatatgttctggcttctggtgtcttacgagagccccgcaaagcatccaaccataacagacgaagaacgtaggtacatagaagagagcatcggggagagcgcaaatctgttaggagcaatggagaaattcaagaccccatggaggaagtttttcacatccatgcccgtctatgcgataattgttgcaaacttctgcaggagttggactttttatttactgctcatcagtcaaccagcttatttcgaggaggtttttggatttgaaatcagcaaggttggcatgttgtctgcggtcccacacctggtcatgacaatcattgtgcctatcggggggcaaattgcagactttctaaggagcaagcaaattctttcaacaactacagtgcgaaagatcatgaactgcgggggttttggcatggaagccacactgcttctggttgttggctactctcatactagaggggtggccatctccttcttggtgcttgcagtgggattcagtggatttgctatctctggtttcaatgtgaaccacttggatattgccccgagatatgccagtatcttaatgggcatttcaaatggtgttggcacgctgtcgggaatggtctgcccgatcattgttggtgcaatgacgaagaacaagtcccgtgaagaatggcagtatgtcttcctcatcgctgcactggtccactatggtggagtcatattttatgcactatttgcctcaggagagaagcaaccttgggcagaccctgaggaaacaagcgaagaaaagtgtggcttcattcatgaagatgaactggatgaagaaacgggggacatcactcagaattacataaattacggtaccaccaaatcctacggcgccacctcacaggagaacggaggctggcctaacggctgggagaaaaaggaagaatttgtgcaagaaagtgcgcaagacgcgtactcctataaggaccgagatgattattcataacgaagctagttgctggattcctttgtagtgtttgtgattaaattaattgtgattgcacaaaatcattttaagaaatgtggtgtaaacatgtaaacacatcaaccaagcaagtcttgctgttcaaaaaataataataatatgaattcaaaacagaccgtgagagtcccatcaagtgcaatctgtggcggcagtcacgtgacgccatttccattcaggccattcgtccttttcgtttgtgatttaaaggtttcctgtagaaataagtaggtattcgttggatccatcaccacgttagagagtacaactacaacagttggcacatgtcatcctacggaagttaggaagccaaagctactggattatgtgaactgcatttatttatttattacactggactgcaaaatatcccagggaaatcctgtctagagacatagtagaactggaaagatggctagattgggtactgacgataatcattgtgtgtatatcatggagtggctatatcttttaattggagaactatattgtatagctagcaaaattgtactgaattattactaggagtgcacagtgtgtgatattttgtgatcttccaaaagcttatcttgcagtgttttgtgaaacgcttgggcacaaacacttatttttatgaacaagagcttgtaaagggaggagtatgctccatgctctcccattcactacctgacagtatcaaaccttcacatttcaatgaaatccaacgtccatgtaacatatcacatgactttttttgcaaaaaagaatataagaagaaatagacttcaatgtattttttattacaactttgtactggttgtaacttgcattaggaaaaatgattaatatatgtataatcgtaaagaatctaataaaaatttactatgaagatatagcccttaaaatgcaatattaacaacaaaaatatatagaaaatttagataatcttccttgataactagagactatatggaactcacaccacaaagctatatataatatgaaaagataaacaatagagattgtatatgtagacgattttatgacctaatgtcccatttaagaggtatttgtcttgagtatatagtacaaagtatattaaaattatatctacatccctgtatatcttatacatatccactcacacaaacataacaaatacttttcacacagaaccaaaaacaagcatacacctaatgttgggtttggggattgcaatttctactttcatagagtcatagaattttagatgggaaaaaaaaaggcattttgctcgtcatttcttaatataattaattcaacaggaactgcaacatttgtgtaccaagcaataagtgcgaagcataaacctgctgtgtgtaaactatccccatactgcttgtggtagcactgatttctttcttttaaagaacttaacatcggagctctttacaatgttttgcgctgataagaatgcacatcccaatttaacgcaaagtgtcacctggtgtgtttacctgtctgttttgggtatttggtctgtttggtgtcctgtgctcttgactggaggccctgctactgcgaatataaaacgtgaagtttgtttctaaatgcaaaccactcctgaccttaagaaactaaagtccctctctgctttgtgtctccaagtactatcatgtgaccataacccttgctgtgctgagtaaaaagatgtgaactgtcattttgttgctgcgaagcaagtgttaataaaatgttctatttaaaaaaaaaaaaa

SEQ ID NO: 12 - protein, 582 aa, Rattus norvegicus
MESVKQRILAPGKEGIKNFAGKSLGQIYRVLEKKQDNRETIELTEDGKPLEVPEKKAPLCDCTCFGLPRRYIIAIMSGLGFCISFGIRCNLGVAIVDMVNNSTIHRGGKVIKEKAKFNWDPETVGMIHGSFFWGYIITQIPGGYIASRLAANRVFGAAILLTSTLNMLIPSAARVHYGCVIFVRILQGLVEGVTYPACHGIWSKWAPPLERSRLATTSFCGSYAGAVIAMPLAGILVQYTGWSSVFYVYGSFGMVWYMFWLLVSYESPAKHPTITDEERRYIEESIGESANLLGAMEKFKTPWRKFFTSMPVYAIIVANFCRSWTFYLLLISQPAYFEEVFGFEISKVGMLSAVPHLVMTIIVPIGGQIADFLRSKQILSTTTVRKIMNCGGFGMEATLLLVVGYSHTRGVAISFLVLAVGFSGFAISGFNVNHLDIAPRYASILMGISNGVGTLSGMVCPIIVGAMTKNKSREEWQYVFLIAALVHYGGVIFYALFASGEKQPWADPEETSEEKCGFIHEDELDEETGDITQNYINYGTTKSYGATSQENGGWPNGWEKKEEFVQESAQDAYSYKDRDDYS

SEQ ID NO: 13 - DNA, 2528 bp, Mus musculus
ggcacgaggctcagtcttaattccactctgccactctgccgcagagcacaattacgccggcgcgatgggaggagaccatgttaaggcaggaagctaacagcagccgctcacctgaggcctaggaagctcccaagggttctgagagctatgagctctgatcagcaaagtcaccattttagacagtagggttcttttgcttttttccttacacaagggttcgatgacgtttctggtcaatctgattaaaagacagcggatttgattgcgataagacttcaaaaccgggaatttacgttgtttttcggtgaggtgacttccagaacagggactcatcagcacccgcccaaataccacggcactgcgcgcgccctcggccaccggatcctccccttccaatgagactttgtgactgtgtgtaccaattctcctattaggaaacccgtgggctgcatgcagctattctgttgtactctctttctcgccctccctcccctctccaactcacagccttgctggaaagctcacctctgctgagaagaaaaagctctaccttaaccaactaagactatgcgcagaatccgtctttcatagccacaacaatttaaatctggtaaggctggacaccagtctttacaagaatggagtcggtaaaacaaaggattttggccccggggaaagaggggataaagaattttgctggaaaatccctcggacagatctacagggtgctggagaagaagcaggacaaccgagagaccatcgagctgacagaggacggtaagcccctggaggtgcctgagaagaaggctccgctatgcgactgcacgtgcttcggcctgccgcgccgctacatcatagccatcatgagcggcctcggcttctgcatatccttcggcatccgctgtaacctgggcgtggccatcgtggacatggtcaacaacagcactatccaccgcggaggcaaagttatcaaggagaaagccaaatttaactgggaccccgagaccgtggggatgatccacggatcgttcttctggggctatatcatcacccagattccaggaggatatatcgcatcgcggctggctgctaaccgggtctttggggctgcgatactgctcacctctaccctcaatatgctgatcccatctgcagccagagtgcattatggatgtgtcatctttgttaggatattgcaaggacttgtggagggtgtcacctacccagcctgtcatgggatatggagcaagtgggcccctcccttggagaggagtaggttggctacaacctccttttgtggttcctatgctggagcagtcattgcaatgcccttagctggtatccttgtgcagtacactggatggtcgtcagtattttatgtgtatggaagctttggcatggtctggtacatgttctggcttctggtgtcttatgagagccctgcaaagcatcctaccattacagatgaagaacgtaggtacatagaggagagcattggagagagcgcaaatctgctaggtgcaatggaaaaatttaagaccccatggaggaagtttttcacatccatgcccgtctacgcgataattgttgccaacttctgcaggagctggactttttatttactgctcatcagtcagccagcttattttgaggaggtttttggatttgaaatcagcaaggttggcatgttgtctgcagtccctcaccttgtcatgacaatcattgtgcctatcggggggcaaattgcagatttcctaaggagcaagcaaattctctcaacaactacagtgagaaagatcatgaattgtgggggttttggcatggaagccacgctgcttctggttgttggctactctcatactagaggggtggccatctccttcttggtgcttgcagtaggattcagtggatttgctatctctggtttcaatgttaatcacttggatattgctccaagatatgccagtatcttaatgggcatttcaaatggcgttggcacgctgtcggggatggtttgccctatcattgttggtgcaatgacaaagaataagtcccgtgaagaatggcagtatgtcttcctcattgctgcactcgtccactatggtggagtcatattttatgcactatttgcctcaggagagaaacaaccttgggcagaccctgaggaaacaagcgaagaaaaatgtggcttcattcacgaagatgaactggatgaagaaacgggggacatcactcagaattacataaattacggtaccaccaaatcttacggtgctacctcacaggagaatggaggctggcctaacggctgggagaaaaaggaagaatttgtgcaagaaggtgcgcaagacgcgtacacctataaggaccgagatgattattcataacgatgctagttgctggattcatttgtagtgtttgtgaatcaattaattgtgattgcacaaaaataattttaaaaatgtggtgtgaacatgtaaacatatcaaccaagcaagtcttgctgttcaaaaaaaaaaaaaaaaaaa

SEQ ID NO: 14 - protein, 582 aa, Mus musculus
MESVKQRILAPGKEGIKNFAGKSLGQIYRVLEKKQDNRETIELTEDGKPLEVPEKKAPLCDCTCFGLPRRYIIAIMSGLGFCISFGIRCNLGVAIVDMVNNSTIHRGGKVIKEKAKFNWDPETVGMIHGSFFWGYIITQIPGGYIASRLAANRVFGAAILLTSTLNMLIPSAARVHYGCVIFVRILQGLVEGVTYPACHGIWSKWAPPLERSRLATTSFCGSYAGAVIAMPLAGILVQYTGWSSVFYVYGSFGMVWYMFWLLVSYESPAKHPTITDEERRYIEESIGESANLLGAMEKFKTPWRKFFTSMPVYAIIVANFCRSWTFYLLLISQPAYFEEVFGFEISKVGMLSAVPHLVMTIIVPIGGQIADFLRSKQILSTTTVRKIMNCGGFGMEATLLLVVGYSHTRGVAISFLVLAVGFSGFAISGFNVNHLDIAPRYASILMGISNGVGTLSGMVCPIIVGAMTKNKSREEWQYVFLIAALVHYGGVIFYALFASGEKQPWADPEETSEEKCGFIHEDELDEETGDITQNYINYGTTKSYGATSQENGGWPNGWEKKEEFVQEGAQDAYTYKDRDDYS